(19) United States
(12) Patent Application Publication
(10) Pub. No.: US 2009/0221610 A1
(43) Pub. Date: Sep. 3, 2009

(54) Compositions and Methods for Treating Cognitive Disorders

(75) Inventors: Amy F.T. Arnsten, Bethany, CT (US); Min Wang, New Haven, CT (US); David A. McCormick, North Haven, CT (US); Brian P. Ramos, New Haven, CT (US)

Correspondence Address: COLEMAN SUDOL SAPONE, P.C., 714 COLORADO AVENUE, BRIDGE PORT, CT, 06605-1601, US

(73) Assignee: Yale University, new Haven, CT (US)

(21) Appl. No.: 12/086,658

(22) PCT Filed: Jan. 31, 2007
(86) PCT No.: PCT/US07/02527

§ 371 Date: Dec. 22, 2008

Related U.S. Application Data
(60) Provisional application No. 60/763,802, filed on Jan. 31, 2006.

(51) Int. Cl. A61K 31/44; A61P 25/00

(52) U.S. Cl. 514/256

(57) ABSTRACT

The present invention relates to the use of inhibitors or blockers of Iₕ (hyperpolarization-activated cationic current) channels in the treatment of cognitive disorders. In preferred aspects of the present invention, an effective amount of a compound is administered to a patient in need, wherein the compound has the chemical structure: Where R₁ is H, or an optionally substituted C₁-C₃ alkyl, preferably a C₂ alkyl (ethyl) group; R² is an optionally substituted C₁-C₃ alkyl group, preferably a methyl group; R³ is H, an optionally substituted C₁-C₃ alkyl (preferably methyl), a halogen or 0(Ci-Ca) alkyl; R⁴ is an optionally substituted C₁-C₆ alkyl, C(O)—(C₁-C₅)alkyl, C(O)-aryl, C(O)O—(C₁-C₄)alkyl, C(O)O-aryl, or an optionally substituted heterocyclic, aryl or heteroaryl group; R⁴ is H or an optionally substituted C₁-C₆ (preferably a C₁-C₃) alkyl; R⁵, R⁶ and R⁷ are each independently H, halogen, an optionally substituted C₁-C₆ alkyl (preferably, an optionally substituted C₁-C₃ alkyl), 0-(C₁-C₃) alkyl, or an optionally substituted heterocyclic, aryl or heteroaryl group; Y⁻ is an anion of a pharmaceutically acceptable salt (a physiologically acceptable anion, preferably a Cl⁻, Br⁻, I⁻, OAc⁻); or a solvate or polymorph thereof, optionally, in combination with guanfacine and/or chelerythrine, and a pharmaceutically acceptable carrier, additive or excipient to a patient in need of therapy.

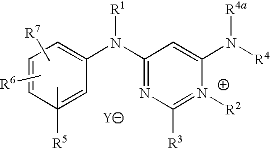

### FIELD OF THE INVENTION

The present invention relates to the use of inhibitors or blockers of Iₕ (hyperpolarization-activated cationic current) channels in the treatment of cognitive disorders.

### BACKGROUND OF THE INVENTION

Hyperpolarization-activated cationic current channels (Iₕ) were initially identified in cardiac myocetes and photoreceptors. Brown, et al., Nature, 280, 235-236 (1979), Brown and DiFrancesco, J. Physiol., and Bader, et al., J. Physiol., 2961-216 (1979). The currents are characterized by permeability to both K⁺ and Na⁺, and modulation by direct binding of cAMP, which is needed for channel opening and shifts activation to more positive channels. DiFrancesco and Tortora, Nature, 351, 145-147 (1991).

The prefrontal cortex (PFC) regulates human behavior using working memory, inhibiting inappropriate impulses and reducing distractibility (Goldman-Rakic, Phil Trans R Soc London, 351: 1445-1453, 1996; Robbins, Phil Trans R Soc London, 351: 1463-1471, 1996). The cardinal symptoms of attention-deficit/hyperactivity disorder (ADHD)—poor attention regulation, impulsivity, and hyperactivity—may all arise from weakened PFC regulation of behavior and thought. The PFC has massive connections to motor and sensory cortices and to subcortical structures such as the caudate and cerebellum. These circuits regulate attention and action, inhibiting inappropriate thoughts and behaviors and coordinating goal-directed actions.

Neuropsychological studies first identified marked impairments in ADHD patients performing tasks requiring PFC function, and imaging studies confirmed both structural and functional insufficiencies in PFC circuits. Attention-deficit/hyperactivity disorder patients also show evidence of genetic alterations, including genes related to catecholamine neurotransmission. See, Arnsten and Li, Biol. Psychiatry, 57, 1377-1384 (2005). PFC deficits have also been observed in a number of other neuropsychiatric disorders (e.g. schizophrenia, bipolar disorder, Posttraumatic Stress Disorder, Anxiety disorders, Tourettes Syndrome), in normal aging, in neurodegenerative disorders such as Alzheimer's and Parkinson's Disease, and following traumatic brain injury to the PFC.

The PFC subserves working memory. Working memory is the ability to bring to mind an event from long term storage, or keep in mind an event that has just occurred, and retain this information in a temporary buffer in order to guide behavior and thought. Given the short term nature of working memory, it cannot involve architectural changes such as structural changes in synapses, as is thought to occur with long term memory consolidation. Rather, working memory is thought to arise from a network of PFC neurons with shared properties, engaged in recurrent excitation. The spatial working memory characteristics of neurons in the primate PFC (area 46) have been well characterized in monkeys performing spatial working memory tasks that require the animal to remember a visually cued spatial location over a brief delay period. The cued position continuously changes, requiring constant updating of spatial working memory. Goldman-Rakic, Neuron 14, 477-485 (1995) identified PFC microcircuits with spatially tuned mnemonic activity during the delay period: pyramidal neurons with similar spatial tuning properties mutually exciting each other, and those with dissimilar spatial properties inhibiting each other via GABAergic interneurons. The horizontal connectivity of layer III pyramidal cells is thought to provide the anatomical basis for PFC microcircuits (Kritzer, J Comp Neurol., 359:131-143, 1995 and Goldman-Rakic, Neuron 14, 477-485 (1995)). These connections allow PFC neurons to continue firing during the delay period when no stimulus is available in the environment, maintaining representations over time even in the presence of distracting stimuli (Miller, et al., J. Neurosci. 16:5154-5167, 1996). This is a fragile process that is highly dependent on the correct neurochemical environment.

Catecholamines have an essential influence on PFC spatial working memory functions. Extensive depletion of catecholamines in PFC (area 46) is as devastating as removing the cortex itself (Brozoski, et al., Science, 205:929-931, 1979). Although early work focused on dopamine actions, it is now known that norepinephrine (NE) has a critical influence via post-synaptic a2A-adrenoceptors (Arnsten and Goldman-Rakic, Science, 230:1273-1276, 1985; Franowicz, et al., J. Neurosci, 22:8771-8777, 2002), and that blockade of these receptors in PFC profoundly impairs spatial working memory (Li and Mei, Behav Neural Biol, 62:134-139, 1994) and erodes delay-related firing (Sawaguchi, J Neurophysiology, 80:2200-2205, 1998; and Li, et al, Neuropsychopharmacol. 21:601-610, 1999). Conversely, stimulation of post-synaptic a2A-adrenoceptors strengthens PFC cognitive functions in mice (Franowicz, et al., J Neurosci, 22:8771-8777, 2002), rats (Tanila, et al., Brain Res Bull 40:117-119, 1996), monkeys (Arnsten and Goldman-Rakic, Science, 230:1273-1276, 1985; Arnsten, et al, J Neurosci, 8:4287-4298, 1988; Cai, et al., Brain Res., 614:191-196, 1993; Rama, et al., Pharmacol Biochem Behav., 54:1-7, 1996; Mao, et al., Biol Psychiatry, 46:1259-1265 1999; Wang et al., Brain Res. 1024:176-182, 2004) and humans (Jakala, et al., Neuropsychopharmacol, 20:119-130, 1999b; Jakala, et al., Neuropsychopharmacology, 20:460-470, 1999a). On the basis of this research in animals, the a2A-adrenoceptor agonist guanfacine is currently in use for treating PFC cognitive deficits in patients with Attention Deficit Hyperactivity Disorder (Hunt, et al., Amer Acad Child Adoles Psychiatry, 34:50-54, 1995; Scahill, et al., Amer J Psychiatry, 158:1067-1074, 2001; Taylor and Russo, J Clin Psychopharm 21:223-228, 2001), Tourettes Syndrome (Scahill, et al., Amer J Psychiahy, 158:1067-1074, 2001) and mild traumatic brain injury involving the PFC (McAllister, et al., Brain Inj., 18:331-350, 2004). Guanfacine is also being tested in patients with schizophrenia, pervasive development disorders and post-traumatic stress disorder. However, the molecular events underlying these therapeutic effects have not been known.

a2A-Adrenoceptor stimulation improves working memory via suppression of cAMP intracellular signaling, consistent with a2A-adrenoceptor coupling with Gi proteins. In contrast to long term memory consolidation processes which are strengthened by cAMP signaling, working memory performance is impaired by infusions of the cAMP analog, Sp-cAMPS, into the rat PFC (Taylor, et al., J Neuroscience (Online) 19:RC23, 1999). Similarly, the working memory performance of aged monkeys is impaired by systemic administration of the PDE4 inhibitor rolipram, which increases endogenous levels of cAMP (Ramos et al., Neuron, 40:835-845, 2003, Ramos et al., Learning and Memory, zxc: zxc, 2006). Recently, the enhancing effects of guanfacine have been blocked by co-infusion of Sp-cAMPS into the rat PFC (Ramos et al, Learning and Memory, zxc: sac, 2006), using low doses of Sp-cAMPS that had no effect on their own (Ramos et al, Neuron, 40:835-845, 2003). Conversely, inhibition of cAMP actions with Rp-cAMPS infusions into rat PFC improved working memory performance similar to findings with guanfacine (Ramos, et al., Neuron, 40:835-845, 2003).

cAMP signaling impairs PFC cognitive operations at the cellular level as well. Recent evidence from in vitro recordings of neurons from PFC slices indicate that cAMP may reduce network connectivity by opening Iₕ channels, thus lowering membrane resistance and functionally weakening cortical connectivity (David McCormick, unpublished data). 1 h channels have been shown to have important effects on dendritic integration in hippocampus, where they are localized on the distal dendrites of pyramidal cells (Nolan, et al., Cell, 119:719-732, 2004). The inputs to hippocampal CA1 pyramidal cells are segrated such that the Schaeffer collaterals from CA3 neurons arrive on the distal portion of the dendrite, while perforant pathway connections from entorhinal cortex terminate more proximally. The opening of Iₕ channels on the distal portion of CA1 dendrites thus functionally disconnects pyramidal cells from CA3 inputs without influencing perforant path connections (Nolan, et al., Cell, 119:719-732, 2004).

As in hippocampus, the HCN1 and HCN2 subunits of the Iₕ channels are both present in PFC, and likely form heteromers that are highly responsive to cAMP (Chen, et al., J Gen Physiol., 117:491-504, 2001 and Ulens and Tytgat, J Biol. Chem., 276:6069-6072, 2001). Electron microscopic studies have noted a2A-adrenoceptors in post-synaptic spines in monkey PFC (Aoki, et al., Brain Res., 650:181-204, 1994; and Aoki, et al., Cerebral Cortex, 8:269-277, 1998).

HCN1 labeling can similarly be observed in the spines of pyramidal cells in monkey PFC, and indeed, recent EM analyses have shown that HCN1 channels are co-localized with alpha-2A-adrenoceptors in the dendritic spines of PFC pyramidal cells. Electrophysiological and cognitive experiments examined whether a2A-adrenoceptor agonists such as guanfacine may improve PFC cognitive function by reducing cAMP and closing Iₕ channels, thus strengthening the PFC networks that underlie delay-related cell firing in monkeys performing a spatial working memory task Parallel studies examined whether infusion of the HCN channel blocker, ZD7288, into the rat PFC would improve behavioral measures of working memory performance.

### BRIEF DESCRIPTION OF THE FIGURES

FIG. 1 shows the effects of prefrontal infusions of the Iₕ channel blocker ZD7288 (ZD; 0.0001 μg) on spatial working memory performance in rats. Results represent mean+/−SEM percent correct on the delayed alternation task. Co-infusion of the cAMP analog 0.21 nmol Sp-cAMPS (Sp) reversed the effects of ZD. ** significantly different than saline (SAL); † significantly different than ZD alone.

FIG. 2 shows the effects of prefrontal infusions of the α2A agonist guanfacine (GFC; 0.0001 μg) on spatial working memory performance in aged rats (n=9). Results represent mean+/−SEM percent correct on the delayed alternation task Co-infusion of 0.21 mmol Sp-cAMPS (Sp) reversed the effects of guanfacine. ** significantly different than saline (SAL); † significantly different than guanfacine alone. Aged animals are particularly sensitive to the enhancing effects of guanfacine and thus were the focus of this study.

FIG. 3 shows the paradigm used for electrophysiological recordings in monkeys performing a WM task (A) ODR task: Trials began when the monkey fixated on a central point for 0.5 sec. A cue was present in 1 of 8 possible locations for 0.5 sec and was followed by a delay period of 2.5 sec. When the fixation point was extinguished, the monkey made a saccade to the location of the remembered cue. (B) Position of the cylinder (big circle) and the region of electrophysiological recording in dorsolateral PFC (red area). PS: principal sulcus; AS: arcuate sulcus. (C) Neuronal activity of a PFC cell on the ODR task. Rasters and histograms are arranged to indicate the location of the corresponding cue. This cell exhibited significant delay-related activity for the 180° location (preferred direction) but not for other directions (e.g. 0°, nonpreferred direction).

FIG. 4 shows the effects of β2A-AR stimulation and blockade on spatially tuned delay-related firing of PFC neurons in monkeys performing a spatial WM task. (A). Iontophoretic application of the β2A-AR agonist, guanfacine, enhanced spatially tuned, delay-related firing for a neuron with weak tuning under control conditions. Rasters and average histograms of neuron H636 during the control condition vs. guanfacine iontophoresis for preferred and nonpreferred directions are shown. (B) Iontophoresis of a low dose of guanfacine increased delay-related firing in a well-tuned neuron as well, while a high dose was without effect. (C) Iontophoresis of the β2-AR antagonist, yohimbine, decreased delay-related firing and eroded spatial tuning of a PFC neuron. (D) Co-iontophoresis of yohimbine with guanfacine reversed the enhancing effects of guanfacine on delay-related firing. Average histograms of neuron H653 during the control condition, guanfacine iontophoresis, and guanfacine/yohimbine co-iontophoresis for preferred and nonpreferred directions are shown. (E) Iontophoretic application of guanfacine (red) significantly enhanced spatially tuned delay related activity at the population level (35 neurons). (F) Suppressive effect of yohimbine (red) on spatial delay-related activity at the population level (15 neurons).

FIG. 5 shows that cAMP suppresses the spatially tuned, delay-related firing of PFC neurons. (A) Iontophoresis of the cAMP analog, Sp-cAMPS, decreased delay-related firing (B) Iontophoresis of the PDE4 inhibitor, etazolate, suppressed delay-related activity. (C) Application of Rp-cAMPS enhanced delay-related activity, and co-iontophoresis of Sp-cAMPS with Rp-cAMPS reversed the enhancing effects of Rp-cAMPS on delay-related firing. (D) Co-iontophoresis of Sp-cAMPS with guanfacine reversed the enhancing effects of guanfacine. (E) Enhancing effect of Rp-cAMPS (red) on spatial delay-related activity at the population level (12 neurons). (F) Suppressive effect of etazolate (red) on spatial delay-related activity at the population level (12 neurons).

FIG. 6 shows the effects of the HCN channel blocker ZD7288 on the spatial WM-related firing and network connectivity of PFC neurons. (A) Iontophoresis of a low dose of ZD7288 increased delay-related firing in a well-tuned neuron. (B) Iontophoretic application of ZD7288 caused a dose-dependent effect on spatially tuned delay-related firing in a neuron with weak tuning under control conditions. ZD7288 at 5 nA and 10 nA enhanced spatially tuned delay-related activity, while a high dose (40 nA) eroded the spatial tuning. (C)Co-iontophoresis of ZD7288 with yohimbine reversed the suppressive effects of yohimbine on delay-related firing. (D) Co-iontophoresis of ZD7288 with etazolate reversed the suppressive effects of etazolate on delay-related firing. (E) Iontophoretic application of ZD7288 (red) significantly enhanced spatially tuned delay related activity at the population level (27 neurons). (F) Co-iontophoresis of ZD7288 with etazolate (green) reversed the suppressing effects of etazolate (red) on delay-related activity at the population level (8 neurons). (G) Bath application of ZD-7288 resulted in a prolongation of the persistent activity of the Up state in ferret PFC cortical slice in vitro as recorded simultaneously with extracellular multiple unit (MU; upper traces) and intracellular recording from layer 5 neurons (lower traces). This effect of ZD7288 was associated with a hyperpolarization of pyramidal neurons and a strengthening of the synaptic barrages mediating the recurrent network activity.

FIG. 7 shows HCN channel expression and manipulation in rat PFC. (A) Rat prelimbic PFC in coronal plane, modified from (Paxinos and Watson, 1997). (B, C) Low (B) and high power (C) light microscopic demonstration of HCN-1 immunoreactivity in PFC. Arrowheads indicate densely labeled apical dendrites in layers II/III. Note that immunoreactivity increases in upper layer processes. Scale bar: 0.4 mm (B); 0.11 mm (C). (D) Blockade of HCN channels in the PFC with infusions of ZD7288 (0.0001 μg/0.51 μl) improved WM performance. The improvement was blocked by co-infusion of the cAMP analog, Sp-cAMPS (0.21 nmol). Results represent mean±SEM percent correct on the delayed alternation task; n−5; * p=0.003 compared to vehicle; † p=0.01 compared to ZD7288. (E) Knockdown of HCN1 channels in the PFC significantly improved WM performance 11-19 days post-transduction compared to scrambled control; n=8; ** p=0.0011 compared to scrambled construct. (F) Representative example of viral expression in the PFC as indicated by β-galactosidase immunohistochemistry, visualized with a secondary antibody conjugated to Alexa Flour® 488. (G) Western blots confirmed shRNA against HCN1 knocks down HCN1 expression in cellular assays.

FIG. 8 shows the expression and co-expression patterns of HCN channels (double arrowheads) and α2A-ARs (arrowheads) in dendritic spines of primate PFC. HCN (A, B) and α2A-AR (C) immunoparticles mark extrasynaptic membranes in the spine head and neck region (compare B to C); curved arrows point to emerging spines. In dual labeling, α2A-AR was visualizd with immunoperoxidase and HCN1 with silver-enhanced nanogold (C) or with reversal of the immunocytochemical sequence (E). Sequential gold-enhancement of nanogold is shown in (F); (G) depicts the reverse procedure as in (E).

HCN channels and α2A-ARs are co-expressed at exrasynaptic sites (E, F), including the spine neck (G), or where a dendrite tapers outwards, possibly to give rise to a spine (1)). Singly labeled profiles in (ID) attest to the specificity of dual immunolabeling; lead counterstaining was omitted to facilitate detection. Asterisks mark spine apparata. ax, axon; den, dendrite; gl, glial process; sp, spine Scale bars: 200 nm.

FIG. 9 shows a model of α2A-cAMP-HCN1 regulation of PFC microcircuits, whereby HCN channel opening shunts inputs to dendritic spines and reduces network activity. α2A-AR stimulation inhibits the production of cAMP and increases the efficacy of cortical inputs.

### BRIEF DESCRIPTION OF THE INVENTION

The present invention relates to a number of compounds which may be used to modulate Iₕ channels, preferably including blocking or inhibiting Iₕ channels to strengthen the connectivity of PFC microcircuits or neurons (prefrontal cortical function) to treat impairment of cognitive function in a patient. Applicants have discovered in animals studies that a compound which functions as an Iₕ channel blocker may be used to enhance prefrontal cortical function and to be useful to treat cognitive deficiencies, the impairment of cognitive function or to enhance cognitive function in a human, including memory disorders/deficiencies and learning disorders/deficiencies, which may result from aging, trauma, stroke, neurotoxic agents, neurodegenerative disorders or anxiety disorders, including those which are associated with drug-induced states, neurotoxic agents, Alzheimer's disease, and aging. These conditions are readily recognized and diagnosed by those of ordinary skill in the art and treated by administering to the patient an effective amount of one or more compounds according to the present invention. The present invention is also useful for the treatment of memory disorders/deficiencies and/or learning disorders/deficiencies which may be associated with such conditions as attention deficit disorder (ADD), attention deficit disorder with hyperactivity (ADD-HD), autism, and pervasive development disorder (PDD, including PDD-NOS).

In addition, Iₕ inhibitors or blockers according to the present invention may be used to treat the loss of function in patients from cAMP opening of Iₕ (HCN) channels and prefrontal cortical impairment (PFC), which are implicated in schizophrenia or certain mood disorders such as depressive disorders. These inhibitors or blockers therefore may be used to treat schizophrenia, and mood disorders, including bipolar disorder, unipolar disorder, dysthymic disorder, post-partum depression, seasonal affective disorder and depression (major depression or major depressive disorder).

Thus, in the present invention, a method of treating cognitive disorders in a patient in thereof comprises administering an effective amount of an inhibitor or blocker of an 1 h channel in said patient. In preferred aspects of the present invention, the Iₕ channel is HCN1, HCN2 or a heteromer of HCN1 or HCN2. A particularly preferred target of the blockers or inhibitors of the present invention is the Iₕ channel comprising a heteromer of HCN1 and HCN2.

In certain preferred aspects, the present invention relates to a compound or method using said compound comprising administering an effective amount of a compound according to the structure:

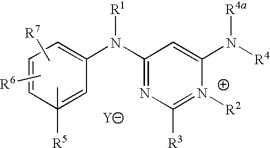

Where R¹ is H, or an optionally substituted C₁-C₃ alkyl, preferably a C₂ alkyl (ethyl) group;
R² is an optionally substituted C₁-C₃ alkyl group, preferably a methyl group;
R³ is H, an optionally substituted C₁-C₃ alkyl (preferably methyl), a halogen or O(C₁-C₃) alkyl;
R⁴ is an optionally substituted C₁-C₆ alkyl, C(O)—(C₁-C₅)alkyl, C(O)-aryl, C(O)O—(C₁-C₄)alkyl, C(O)O-aryl, or an optionally substituted heterocyclic, aryl or heteroaryl group;
R^{4a} is H or an optionally substituted C₁-C₆ (preferably a C₁-C₃) alkyl;
R⁵, R⁶ and R⁷ are each independently H, halogen, an optionally substituted C₁-C₆ alkyl (preferably, an optionally substituted C₁-C₃ alkyl), O—(C₁-C₃) alkyl, optionally substituted heterocyclic, aryl or heteroaryl group;
Y⁻ is an anion of a pharmaceutically acceptable salt (a physiologically acceptable anion, preferably a Cl⁻, Br⁻, I⁻, OAc⁻);
or a solvate or polymorph thereof, optionally, in combination with a pharmaceutically acceptable carrier, additive or excipient to a patient in need of therapy.

In certain preferred aspects of the present invention, the compound to be used in the present methods is ZD7288, represented by the formula presented hereinbelow.

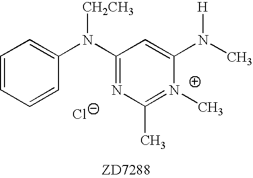

Additional aspects of the present invention relate to the administration of an Iₕ channel blocker according to the present invention in combination therapy, for example, with an effective amount of another agent, such as an agent which inhibits cAMP such as guanfacine (N-(diaminomethylidene)-2-(2,6-dichlorophenyl)acetamide) or its pharmaceutically acceptable salt and/or chelerythrine, in its neutral or salt form (which includes all pharmaceutically acceptable salt forms, including the naturally occurring chloride salt form), preferably chelerythrine chloride, in order to potentiate the activity of ZD7288 or one of its analogs or derivatives, thereof.

### DETAILED DESCRIPTION OF THE INVENTION

As used herein, the following terms have the following respective meanings. Other terms that are used to describe the present invention have the same definitions as those generally used by those skilled in the art. Specific examples recited in any definition are not intended to be limiting in any way.

The term “patient” or “subject” refers to a mammal, preferably a human to which one or more of the present methods is applied.

The term “effective” is used in context, to describe an amount of a compound or compound, a component or components or a substance or substances which produce a result intended from the use of that compound(s), component(s) or substance(s).

The term “compound”, as used herein, unless otherwise indicated, refers to any specific chemical compound disclosed herein. The compounds of the present invention include all stereoisomers (i.e, cis and trans isomers), tautomers, and all optical isomers of the present compound and related analogs in context (eg., R and S enantiomers), as well as racemic, diastereomeric and other mixtures of such isomers, as well as all solvates and polymorphs of the compounds.

The term “Iₕ channel” refers to a channel formed from one or more hyperpolarization-activated cAMP-regulated cation (HCN) channels. See, Chen, et al., J. Gen. Physiol., 117, 491-503 (May, 2001). Recently, a family of four mammalian genes encoding hyperpolarization-activated cAMP-regulated cation (HCN) channels were cloned. Santoro, et al., Proc. Natl. Acad. Sci. USA, 94, 14815-14820, 1997 and Santoro, et al., Cell, 93, 717-729, 1998. The four genes (H1, H2, H3 and H4) encode highly similar proteins that belong to the voltage-gated K channel superfamily and contain six transmembrane segments, a pore-forming P region and cytosolic NH₂ and COOH termini. Jan and Jan, Annu. Rev. Neurosci., 20, 91, (1997). The COOH terminus of the HCN channels also contains a cyclic nucleotide binding domain (CNBD) homologous to those of other cyclic nucleotide binding proteins, including the cyclic nucleotide-gated channels of photoreceptors and olfactory neurons. See Zagota nd Sigelbaum, Annu. Rev. Neurosci., 19, 235-263 (1996).

In aspects of the present invention, compounds according to the present invention block or inhibit L channels, including any one or more of the HCN subunits 1-4, preferably HCN 1 and/or 2 and in particular, the HCN 1 & 2 heteromer (HCN1/HCN2 heteromer Iₕ channel) as described by Chen, et al., Gem Physiol., 117, 491-503 (May, 2001). HCN 1 and 2 are found in the prefrontal cortex (PFC) and the hippocampus and form a heteromer there. The HCN1/HCN2 heteromer Iₕ channel is the preferred target for blockers to effect treatment of cognitive disorders according to the present invention.

The term “Iₕ channel blocker” “Iₕ channel inhibitor”, “blocker of Iₕ channels” or “inhibitor of Iₕ channels” are used interchangeably throughout the present application to describe compounds which directly inhibit or block at least a significant part of the function of Iₕ channels to generate hyperpolarization-activated cation currents. The term “direct” means that the action of the blocker or inhibitor according to the present invention is directly on the Iₕ channel, rather than inhibition at another site, which indirectly may cause the release of one or more substances which cause inhibition. By reducing or preventing these cation currents from developing, it is shown that Iₕ channel blockers may be used to treat cognitive disorders by enhancing cognitive functions such as memory or learning, among others.

“Alkyl” refers to a monovalent hydrocarbon radical, preferably a folly saturated hydrocarbon group, containing carbon and hydrogen which may be a straight chain, branched, or cyclic. Examples of alkyl groups are methyl, ethyl, n-butyl, n-heptyl, isopropyl, 2-methylpropyl, cyclopropyl, cyclopropylmethyl, cyclobutyl, cyclopentyl, cyclopentylethyl and cyclohexyl. “Cycloalkyl” groups refer to cyclic alkyl groups such as cyclopropyl, cyclobutyl, cyclopentyl and cyclohexyl. C₁-C₆ alkyl groups are preferably used in the present invention. The term “alkyl” also refers to unsaturated alkyls, containing one or more unsaturated groups, within context, and includes alkenyl and alkynyl groups as described hereinbelow, distinguished from aromatic groups, which are fully unsaturated.

The term “substituted” shall mean, within context, a group (substitution group) which is added to a moiety within a compound, e.g. “an optionally substituted alkyl group”. Pursuant to the present invention, within context, the substitution group can be alkyl or alkylene groups containing from 1 to 6 carbon atoms, preferably a lower alkyl containing 1-3 carbon atoms, aryl, substituted aryl, acyl, halogen (i.e., alkyl halos, e.g., CF₃), hydroxy, alkoxy, alkoxyalkyl, amino, alkyl and dialkyl amino, acylamino, acyloxy, aryloxy, aryloxyalkyl, carboxyakyl, carboxamido, thio, thioethers, both saturated and unsaturated cyclic hydrocarbons, heterocycles and the like

“Substituted alkyl” refers to alkyls (including unsaturated alkyls) as above-described which include one or more substituted groups such an alkyl or alkylene groups containing from 1 to 6 carbon atoms, preferably a lower alkyl containing 1-3 carbon atoms, aryl, substituted aryl, acyl, halogen (i.e., alkyl halos, e.g., CF₃), hydroxy, alkoxy, alkoxyalkyl, amino, alkyl and dialkyl amino, acylamino, acyloxy, aryloxy, aryloxyalkyl, carboxyalkyl, carboxamido, thio, thioethers, both saturated and unsaturated cyclic hydrocarbons, heterocycles and the like. The term “substituted cycloalkyl” has essentially the same definition as and is subsumed under the term “substituted alkyl” for purposes of describing the present invention.

“Aryl” refers to a substituted or unsubstituted monovalent aromatic radical having a single ring (e.g., phenyl) or multiple condensed rings (e.g., naphthyl). Other examples include heterocyclic aromatic ring groups having one or more nitrogen, oxygen, or sulfur atoms in the ring, such as imidazolyl, furyl, pyrrolyl, pyridyl, thienyl and indolyl, among others. Therefore, “aryl” as used herein includes “heteroaryls” having a mono- or polycyclic ring system which contains 1 to 15 carbon atoms and 1 to 4 heteroatoms, and in which at least one ring of the ring system is aromatic. Heteroatoms are sulfur, nitrogen or oxygen.

“Substituted aryl” refers to an aryl as just described that contains one or more functional groups such as lower alkyl acyl, aryl, halogen, alkylhalos (e.g., CF₃), hydroxy, alkoxy, alkoxyalkyl, amino, alkyl and dialkyl amino, acylamino, acyloxy, aryloxy, aryloxyalkyl, carboxyalkyl, carboxamido, thio, thioethers, both saturated and unsaturated cyclic hydrocarbons, heterocycles and the like.

“Halo” and “halogen” are used in the conventional sense to refer to a chloro, bromo, fluoro or iodo substituent. The terms “haloalkyl,” “haloalkenyl” or “haloalkynyl” (or “halogenated alkyl”) refers to an alkyl, alkenyl or alkynyl group, respectively, in which at least one of the hydrogen atoms in the group has been replaced with a halogen atom.

“Heterocycle” or “heterocyclic” refers to a carbocylic ring wherein one or more carbon atoms have been replaced with one or more heteroatoms such as nitrogen, oxygen or sulfur. A substitutable nitrogen on an aromatic or non-aromatic heterocyclic ring may be optionally substituted. The heteroatoms N or S may also exist in oxidized form such as NO, SO and SO₂. Examples of heterocycles include, but are not limited to, piperidine, pyrrolidine, morpholine, thiomorpholine, piperazine, tetrahydrofuran, tetrahydropyran, 2-pyrrolidinone, δ-velerolactam, δ-velerolactone and 2-ketopiperazine, among numerous others.

“Heteroatom-containing” refers to a molecule or molecular fragment in which one or more carbon atoms is replaced with an atom other carbon, e.g., nitrogen, oxygen, sulfur, phosphorus or silicon. “Substituted heterocycle” refers to a heterocycle as just described that contains one or more functional groups such as lower alkyl, acyl, aryl, cyano, halogen, hydroxy, alkoxy, alkoxyalkyl, amino, alkyl and dialkyl amino, acylamino, acyloxy, aryloxy, aryloxyalkyl, carboxyalkyl, carboxamido, thio, thioethers, both saturated and unsaturated cyclic hydrocarbons, heterocycles and the like. In other instances where the term “substituted” is used, the substituents which fall under this definition may be readily gleaned from the other definitions of substituents which are presented in the specification as well the circumstances under which such substituents occur in a given chemical compound. One having ordinary skill in the art will recognize that the maximum number of heteroatoms in a stable, chemically feasible heterocyclic ring, whether it is aromatic or non-aromatic, is determined by the size of the ring, degree of unsaturation, and valence of the heteroatoms. In general, a heterocyclic ring may have one to four heteroatoms so long as the heterocyclic ring is chemically feasible and stable.

“Isostere” refers to compounds that have substantially similar physical properties as a result of having substantially similar electron arrangements.

“Amine” refers to aliphatic amines, aromatic amines (e.g., aniline), saturated heterocyclic amines (e.g., piperidine), and substituted derivatives such as an ably morpoline. “Amine” as used herein includes nitrogen-containing aromatic heterocyclic compounds such as pyridine or purine.

“Aralkyl” refers to an alkyl group with an aryl substituent, and the term “aralkylene” refers to an alkenyl group with an aryl substituent. The term “alkaryl” refers to an aryl group that has an alkyl substituent, and the term “alkarylene” refers to an arylene group with an alkyl substituent. The term “arylene” refers to the diradical derived from aryl (including substituted aryl) as exemplified by 1,2-phenylene, 1,3-phenylene, 1,4-phenylene, 1,2-naphthylene and the like.

“Alkenyl” refers to a branched or unbranched hydrocarbon group typically although not necessarily containing from 2 to about 12 carbon atoms and at least one double bond, such as ethenyl, n-propenyl, isopropenyl, n-butenyl, isobutenyl, octenyl, decenyl, and the like. Generally, although again not necessarily, alkenyl groups herein contain 2 to about 12 carbon atoms. The term “lower alkenyl” intends an alkenyl group of two to six carbon atoms, preferably two to four carbon atoms.

“Substituted alkenyl” refers to alkenyl substituted with one or more substituent groups, and the terms “heteroatom-containing alkenyl” and “heteroalkenyl” refer to alkenyl in which at least one carbon atom is replaced with a heteroatom.

“Alkynyl” as used herein is subsumed under the term alkyl and refers to a branched or unbranched hydrocarbon group typically although not necessarily containing 2 to about 12 carbon atoms and at least one triple bond, such as ethynyl, n-propynyl, isopropynyl, n-butynyl, isobutynyl, octynyl, decynyl, and the like. Generally, although again not necessarily, alkynyl groups herein contain 2 to about 12 carbon atoms. The term “lower alkynyl” intends an alkynyl group of two to six carbon atoms, preferably three or four carbon atoms. “Substituted alkynyl” refers to alkynyl substituted with one or more substituent groups, and the terms “heteroatom-containing alkynyl” and “heteroalkynyl” refer to alkynyl in which at least one carbon atom is replaced with a heteroatom.

“Alkoxy” as used herein refers to an alkyl group bound through an ether linkage; that is, an “alkoxy” group may be represented as —O-alkyl where alkyl is as defined above. A “lower alkoxy” group intends an alkoxy group containing one to six, more preferably one to four, carbon atoms.

The term “coadministration”, “coadministered” or “combination therapy” is used to describe a therapy in which at least two active compounds in effective amounts are used to enhance cognitive function or to treat a cognitive deficit using an Iₕ blocker or inhibitor in combination with guanfacine or its pharmaceutically acceptable salt or chelerythrine (as its neutral or salt form) as otherwise described herein at the same time. Although the term coadministration preferably includes the administration of two active compounds to the patient at the same tire, it is not necessary that the compounds be administered to the patient at the same time, although effective amounts of the individual compounds will be present in the patient at the same time.

The term “cognitive function” is used to describe an endeavor or process by a patient or subject that involves thought or knowing. The diverse functions of the association cortices of the parietal, temporal and frontal lobes, which account for approximately 75% of all human brain tissue, are responsible for much of the information processing that goes on between sensory input and motor output. The diverse functions of the association cortices are often referred to as cognition, which literally means the process by which we come to know the world. Selectively attending to a particular stimulus, recognizing and identifying these relevant stimulus features and planning and experiencing the response are some of the processes or abilities mediated by the human brain which are related to cognition. Compounds and compositions of the present invention may be used to enhance cognition or reduce impairment of cognitive function.

Impairment of cognitive function (“cognitive disorder”) includes memory disorders and learning disorders, which are treatable according to the present, including those disorders that result from aging, trauma, stroke, neurodegenerative disorders or anxiety disorders. Examples of neurodegenerative disorders include, but are not limited to, those associated with drug-induced states, neurotoxic agents, Alzheimer's disease, and aging. These conditions are readily recognized and diagnosed by those of ordinary skill in the art and treated by administering to the patient an effective amount of one or more compounds according to the present invention. The present invention is also useful for the treatment of attention deficit disorder (ADD), attention deficit disorder with hyperactivity (ADD-HD), autism, pervasive development disorder (PDD, including PDD-NOS), learning disabilities and disorders associated with PFC dysfunction such as and schizophrenia and bipolar disorder. etc.

“Anxiety disorders” include affective disorders such as all types of depression, bipolar disorder, cyclothymia, and dysthymia, anxiety disorders such as generalized anxiety disorder, panic, phobias and obsessive-compulsive disorder, stress disorders including post-traumatic stress disorder, stress-induced psychotic episodes, psychosocial dwarfism, stress headaches, and stress-related sleep disorders, and can include drug addiction or drug dependence.

An “anion of a pharmaceutically acceptable salt” refers to a negatively charged species otherwise represented as Y⁻ in the structures according to the present invention. The anions of pharmaceutically acceptable salts, include, for example, chloride, bromide, iodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)] anions, among numerous others.

The compositions of the present invention may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally, or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions of this invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically. Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of a compound used in a pharmaceutical composition of the instant invention that may be combined with the carrier materials to produce a single dosage form will vary depending upon the host treated, the particular mode of administration and may vary widely. Preferably, the compositions should be formulated to contain between about 10 milligrams to about 500 milligrams of active ingredient

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

Synthesis of Compounds According to the President Invention

The compounds according to the present invention may be synthesized by synthetic methods which are well-known in the art. The synthesis of compounds according to the present invention may proceed by following the general procedure for such compounds as presented in U.S. Pat. No. 5,223,505, relevant portions of which are incorporated by reference herein, PCT application WO 90/12790, a counterpart application to U.S. Pat. No. 5,223,505 and published Japanese patent application 2004-59465. All of the methods for synthesizing compounds according to the present invention are well-known in the art or are adapted from well-known methods in the art without undue experimentation.

In addition to the patent publication references cited above, compounds according to the present invention may be synthesized by condensing a substituted aniline compound onto a pyrimidine intermediate containing a leaving group (halogen such as Br or I) at the 6 position of a substituted 4-aminopyrimidine ring or related pyrimidine analog. The starting materials for such a synthesis may be purchased commercially or produced using methods well known in the art. Alkylation of the appropriate amino group of the resulting intermediate is effected using one or more standard alkylating agents. The resulting compound may be used directly or the counterion may be changed (ion-exchange) to reflect desired solubility/administration parameters.

The invention is described further in the following examples, which are illustrative and in way limiting.

The present invention illustrates the effects of Iₕ channel blockade in prefrontal cortex on 1) cognitive performance in rats engaged in a spatial working memory task, and 2) firing patterns of prefrontal cortical neurons in monkeys performing a spatial working memory task.

### Example

### Spatial Working Memory Performance in Rats

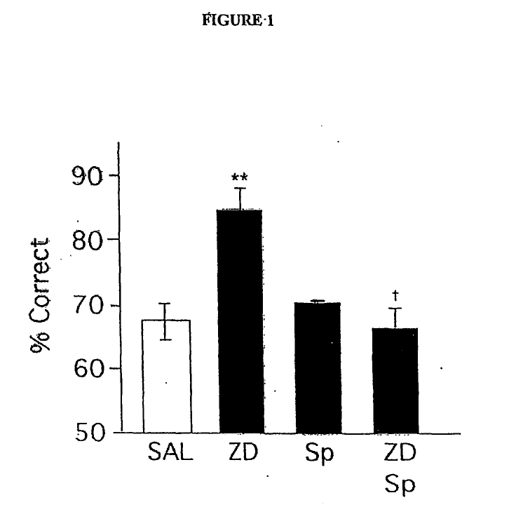

Rats (n=5) were tested on the spatial delayed alternation task in a T maze, a classical test of prefrontal cortical function in rodents (Larsen and Divac, Physiolog Psychol, 6:15-17, 1978). Rats were trained on the task, and then implanted with cannula aimed at the prelimbic prefrontal cortex as described in (Ramos, et al., Neuron, 40:835-845, 2003). Following recovery from surgery, rats were infused with saline (0.5 μl), the Ih channel blocker ZD7288 (0.0001 μg/0.5 μl), the cAMP analog Sp-cAMPS (0.21 nmol/0.5 μl), or a combination of ZD7288 (0.0001 μg)+Sp-cAMPS (0.21 nmol). There was at least one week washout between drug treatments, and a within subjects comparison was performed (2 way analysis of variance with repeated measures with user defined contrasts). As can be seen in FIG. 1, ZD7288 infusions into rat prefrontal cortex significantly improved working memory performance compared to saline control (F(1,4)=42.7, p=0.003). This improvement was significantly reduced by co-administration of the cAMP analog, Sp-cAMPS (F(1,4)=18.5, p=0.013), using a dose of Sp-cAMPS that has no effect on its own (p>0.3).

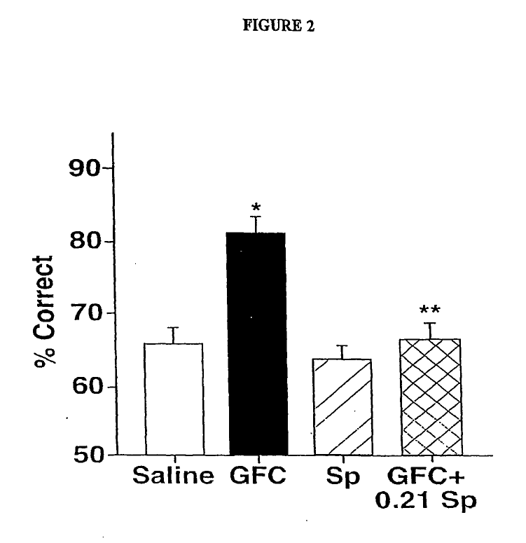

Similar enhancing effects have been observed previously with infusions of the α2A agonist guanfacine into prefrontal cortex of monkeys (Mao et al, Biol Psychiatry 46:1259-1265, 1999) and rats (e.g. FIG. 2). The enhancing effects of guanfacine, like those of ZD72388, can be reversed by co-infusion of Sp-cAMPS (FIG. 2). These results are consistent with the hypothesis that α2A agonists such as guanfacine strengthen prefrontal cortical cognitive function by inhibiting the production of cAMP, which in turn closes Ih channels.

### Further Examples

Spatial working memory is maintained by spatially tuned, recurrent excitation within networks of prefrontal cortical (PFC) neurons, evident during delay periods in working memory tasks. Stimulation of post-synaptic α2A adrenoceptors (α2A-ARs) is critical for working memory. The following examples show that α2A-AR stimulation strengthens working memory through inhibition of cAMP, closing Hyperpolarization-activated Cyclic Nucleotide-gated (HCN) channels and strengthening the functional connectivity of PFC networks. Ultrastructurally, HCN channels and α2A-ARs were colocalized in dendritic spines in monkey PFC. In electrophysiological studies, either α2A-AR stimulation, cAMP inhibition or HCN channel blockade enhanced spatially tuned delay-related firing of PFC neurons in monkeys performing a working memory task. Conversely, delay-related network firing collapsed under conditions of excessive cAMP. In behavioral studies, HCN channel blockade or knockdown of HCN1 channels in rat PFC improved working memory performance. These data reveal a powerful mechanism for rapidly altering the strength of working memory networks in PFC.

Single Neuron Recording and Iontophoresis in Monkeys Performing a Spatial WM task

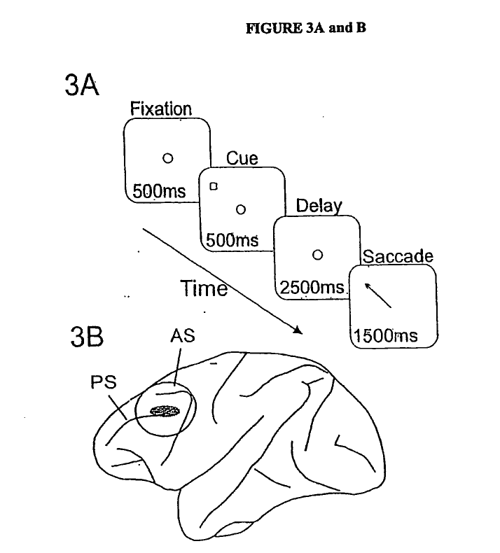

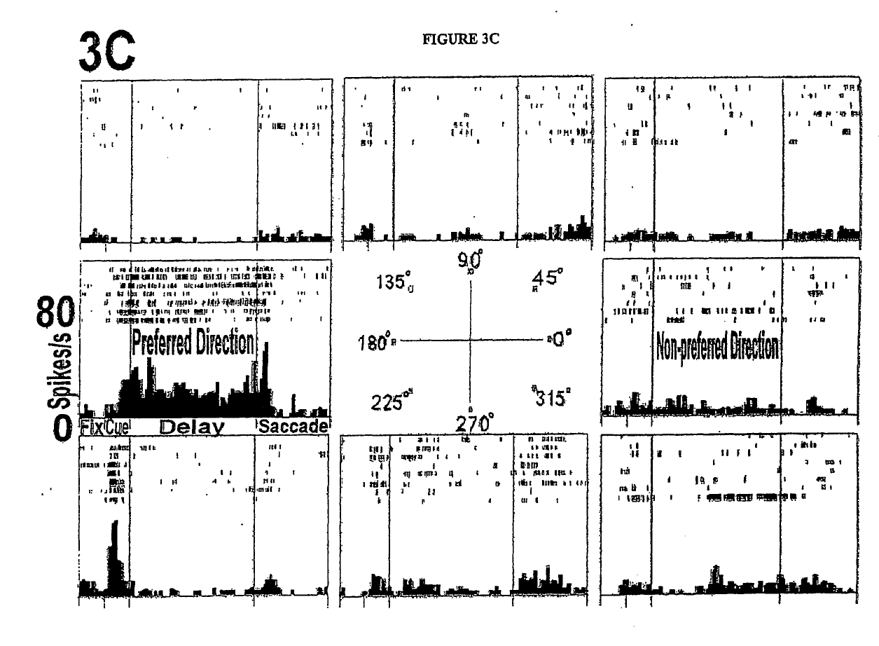

Studies were performed on 4 adult male rhesus monkeys trained on the spatial ODR task (FIG. 3). Iontophoretic electrodes, neuronal recording and drug delivery were as described in (Wang et al., 2004) and also are provided in the Supplemental Material. Guanfacine, yohimbine, ZD7288(Tocris, Ellisville, Mo.) and etazolate (Sigma, St. Louis, Mo.) were dissolved at 0.01M in triple-distilled water (adjusted with HCl to pH 3.5-4.0). Sp-cAMP and Rp-cAMP (Sigma) were dissolved at 0.01M in triple-distilled water (adjusted with NaOH to pH 9). Two-way ANOVA was used to examine the spatial tuned task-related activity with regard to: (1) different periods of the task (cue, delay, response vs. fixation) and (2) different cue locations. One-way ANOVAs were employed to assess the effect of the drug application on cells displaying delay-related activity.

In Vitro Recording from PFC Slices

Methods for extracellular multiple unit and intracellular recording in ferret PFC slices have been detailed elsewhere (Shu et al., 2003). Slices (0.4 mm) from 2-4 month old ferret PFC were maintained in either an interface or submerged chamber (35-36° C.) in a slice solution containing (in mM): NaCl, 126; KCl, 3.1; MgSO₄, 1; NaHPO₄, 1.25; CaCl₂, 1; NaHCO₃, 26; dextrose, 10, and aerated with 95% O₂, 5% CO₂ to a final pH of 7.4. Simultaneous extracellular multiple unit and intracellular recordings were performed in layer 5 with the electrodes approximately 100 mm of one another. Intracellular micropipettes contained 2 M potassium acetate, while whole cell recording pipettes contained KGluconate 140, KCl 3, MgCl₂ 2, Na₂ATP 2, HEPES 10, and EGTA 0.2.

Assessment of Spatial WM Performance in Rats

Male Sprague Dawley rats (240-260 g; Taconic, Germantown, N.Y.) were trained on the delayed alternation task as described in Ramos et al., 2003. Guide cannula were implanted dorsal in prelimbic PFC (AP: +3.2 mm; ML: ±0.75 mm; DV: −4.2 mm). For infusions, needles reached to 4.5 mm DV; infused at 0.25 μL/min for 2 min. Drug or vehicle was administered in a counterbalanced order with at least 1 week between infusions. ZD7288 was dissolved in saline to a dose of 0.0001 μg/0.5 μL. Sp-cAMPS was dissolved in sterile phosphate-buffered saline at 0.21 nmol, a dose with no effect on its own, but sufficient to reverse the effects of guanfacine (Ramos et al., 2006). The experimenter was blind to treatment.

Viral Knockdown of HCN1

The coding region of HCN1 was amplified from rat brain cDNA, cloned into pSTBlue-1 (Novagen, San Diego Calif.), sequenced, and subcloned into pAAV-MCS (Stratagene, La Jolla, Calif.) to create pAAV-HCN1. A vector containing an H1 promoter for shRNA expression and a CMV promoter for lacZ expression flanked by AAV ITR sequences (pAAV-IacZ-shRNA) was created to allow viral expression of shRNAs. Two sets of shRNAs constructs (shRNA-HCN1.1 and shRNA-HCN1.2) directed against HCN1 were generated and tested in HEK293 cells by co-transfection with pAAV-HCN1. A control shRNA viral construct containing a scrambled sequence (shRNA-scrHCN1) was created with an identical nucleotide composition as the shRNA-HCN1.1 target sequence with no homology to any mammalian gene in the Genbank database. AAV2 virus was produced by transfection of HEK293 cells with pAAV-HCN1 or one of the pAAV-IacZshRNA constructs, and pDG essentially as described by (Auricchio et al., 2001). Viral titers were determined by determining viral genome copy number and by infectious titer assays in HEK293 cells.

Rats were implanted with cannula, and trained until achieving baseline performance of ˜70% correct for at least 5 consecutive days. The active or scrambled virus (5 μL) was infused into the prelimbic PFC at a rate of 0.25 μL/min WM was assessed the following day and for the subsequent 3 weeks. Rats were perfused and viral transfection was confirmed in PFC with a monoclonal antibody against B-galactosidase (Mouse anti B-gal 1:400, overnight, RT; Promega Corp., Madison, Wis.) followed by a secondary antibody (Goat anti mouse conjugated to Alexa Flour® 488, 1:400, 4 h, RT; Invitrogen/Molecular Probes, Carlsbad, Calif.). Antibodies were prepared in 0.1M PB containing 0.1% Triton X and 2% normal goat serum. All behavioral studies were performed blind to treatment.

HCN and α2A-AR Localization in Monkey PFC-Electron Microscopic Studies

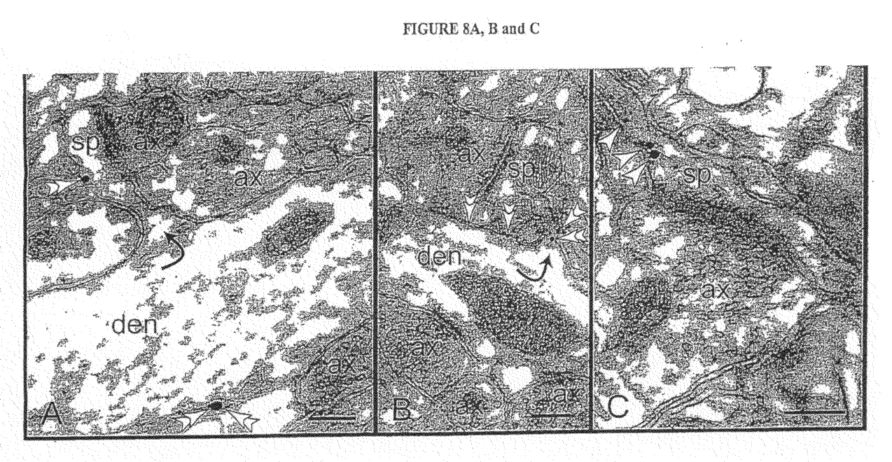

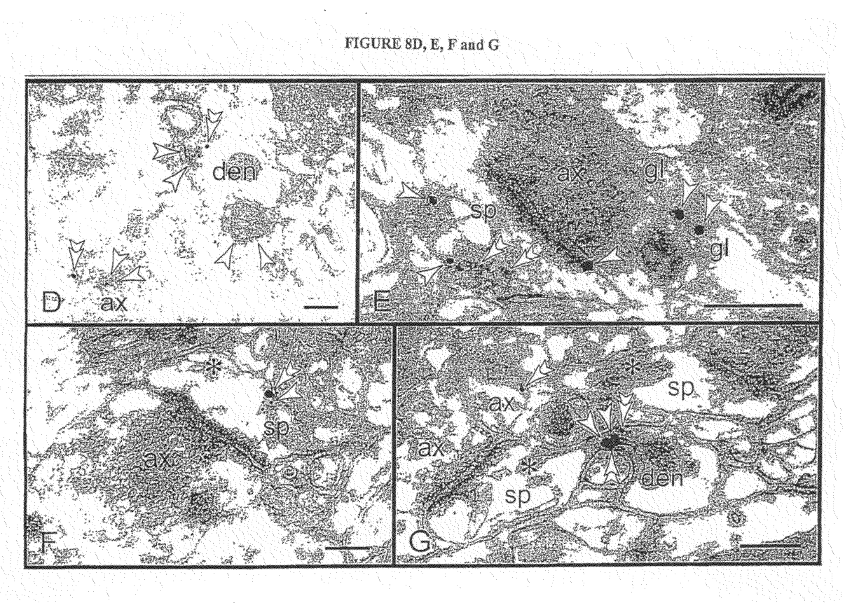

Monkey PFC tissue (n=3) was prepared as described in the Supplemental Material. For single HCN1, HCN-pore or α2A-AR immunocytochemistry, primary antibodies were complexed with nanogold conjugates, either directly or using biotinylated bridging antibodies. F dual immunolabeling, we used combinations of enzymatic and/or gold-based immunotechniques and reversal of the immunocytochemical sequence (FIG. 8D-G). See Paspalas and Goldman-Raki (2004). Sections were processed for electron microscopy and layers I-III of area 46 were sampled for analysis under a JEM 1010 (Jeol, Tokyo, Japan) transmission electron microscope at 80 KV. Immunoreactive structures were digitally captured with a BioScan 792 (Gatan, Pleasanton, Calif.).

### Results

Monkey Physiology: PFC Neurons Exhibit Enhanced Spatial Tuning when cAMP-HCN Signaling is Inhibited by α-2A-ARs

Monkeys performed an oculomotor spatial delayed response (ODR) task, illustrated in FIG. 3A. The ODR task requires the monkey to make a memory-guided saccade to a visuo-spatial target. Stimulus position was randomized over trials to ensure use of WM. Neurons were recorded from area 46 of the dorsolateral PFC (FIG. 3B) as the monkey performed the task. FIG. 3C shows the activity of a PFC neuron with task-related firing. This neuron shows delay-related firing for the 180° location (preferred direction) but not for other directions (e.g. 0°, nonpreferred direction). In the present study, 128 neurons with delay-related mnemonic activity were isolated and subjected to iontophoretic application of pharmacological agents See Table 1, below summarizing the effects of drugs (Table 1) and their reversal (Table 2) on PFC neuronal response.

TABLE 1
Effects of manipulating α2A-AR stimulation,
cAMP signaling, or HCN channel blockade on spatial
delay-related firing of PFC neurons.

| Drug Effect | Increased | | Decreased | | Un-changed |
|---|---|---|---|---|---|
| Guanfacine (5-15 nA) | 28 | (80%) | | | 7 (20%) |
| Guanfacine (20-50 nA) | | | 9 | (100%) | |
| Yohimbine | | | 15 | (100%) | |
| Rp-cAMP | 8 | (67%) | | | 4 (33%) |
| Sp-cAMP | 9 | (33%) | 13 | (48%) | 5 (19%) |
| Etazolate | | | 10 | (83%) | 2 (17%) |
| ZD7288 (5-15 nA) | 19 | (70%) | | | 8 (30%) |
| ZD7288 (20-50 nA) | | | 10 | (100%) | |

TABLE 2
Summary for reversal experiments

| | |
|---|---|
| Reversal | No Reversal |

| | | | |
|---|---|---|---|
| Yohimbine reversal of guanfacine | 6 | (100%) | |
| Sp-cAMP reversal of guanfacine | 6 | (67%) | 3 (33%) |
| Sp-cAMP reversal of Rp-cAMP | 6 | (100%) | |
| ZD 7288 reversal of yohimbine | 5 | (71%) | 2 (31%) |
| ZD 7288 reversal of etazolate | 6 | (75%) | 2 (25%) |

Effects of α2A-AR Stimulation on PFC Neurons with WM Activity

Intra-PFC administration of the α2A-AR agonist, guanfacine, improves WM performance (Mao et al., 1999). However, guanfacine has never been examined for its effects on PFC neuronal firing. In the present study, guanfacine was applied iontophoretically to 35 neurons with spatial delay-related activity. Low doses of guanfacine (5-15 nA) significantly enhanced delay-related activity for the preferred direction in 28 out of 35 cases, while having no effect on neuronal activity of the remaining 7 neurons. In contrast, high doses of guanfacine (20-50 nA) suppressed delay-related activity in 9 out of 9 neurons, perhaps due to stimulation of pre-synaptic α2A-ARs, reducing endogenous NE release.

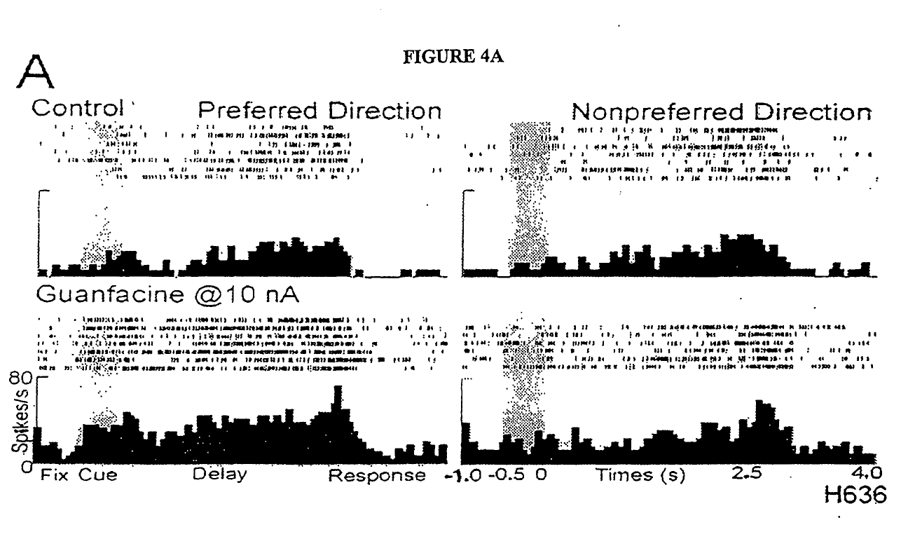

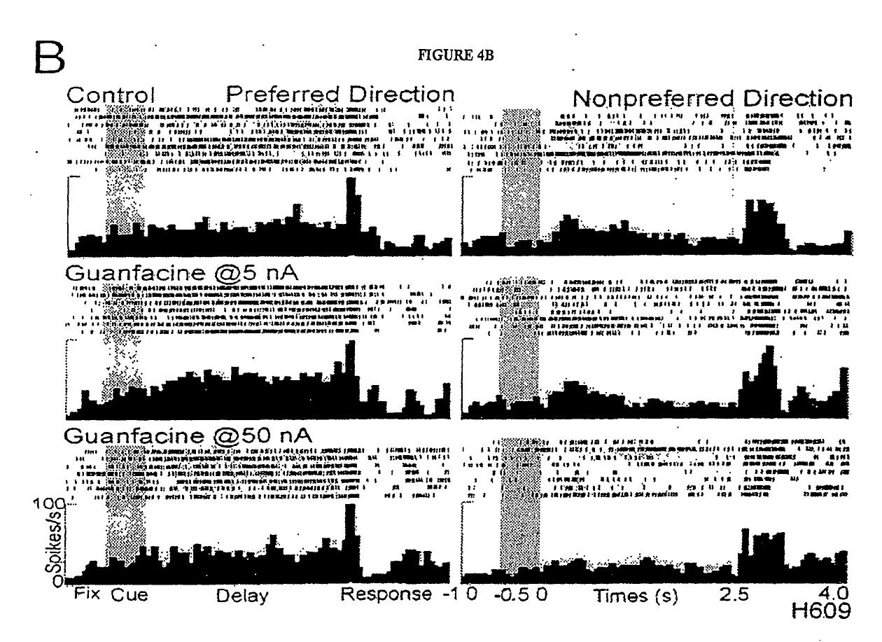

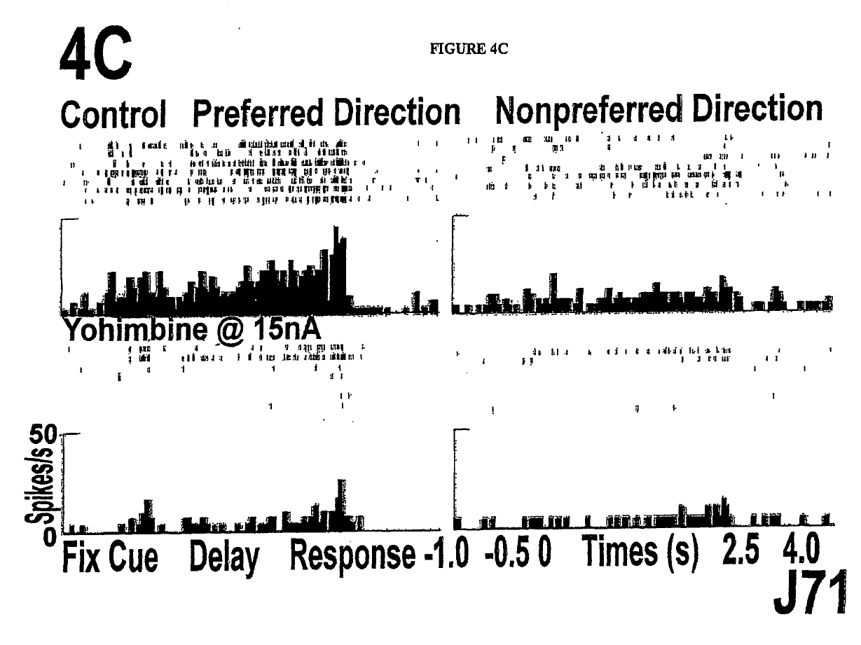

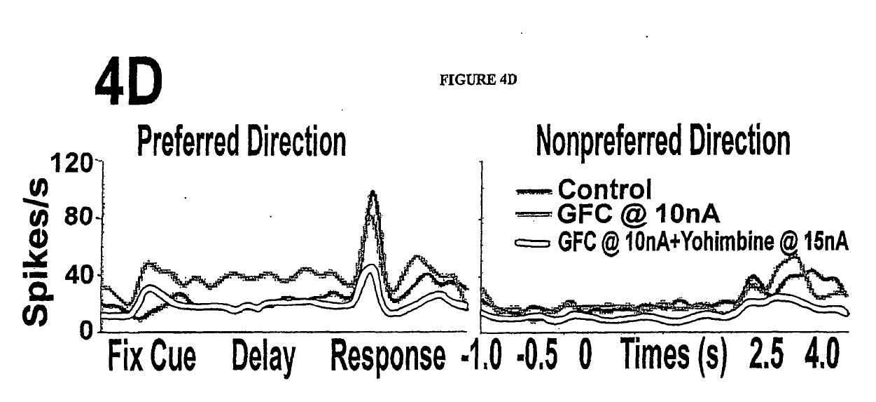

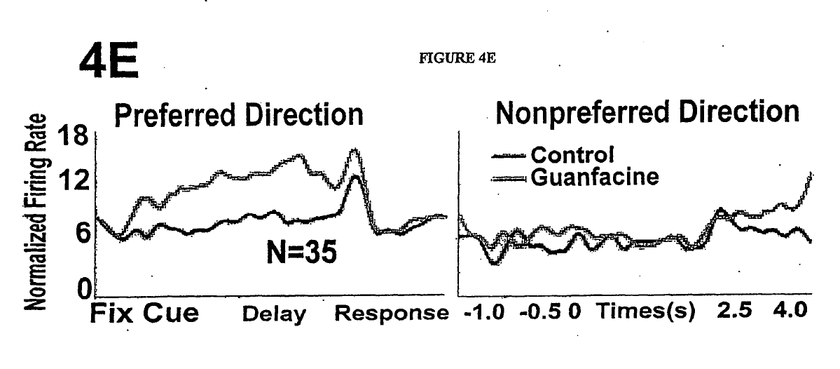

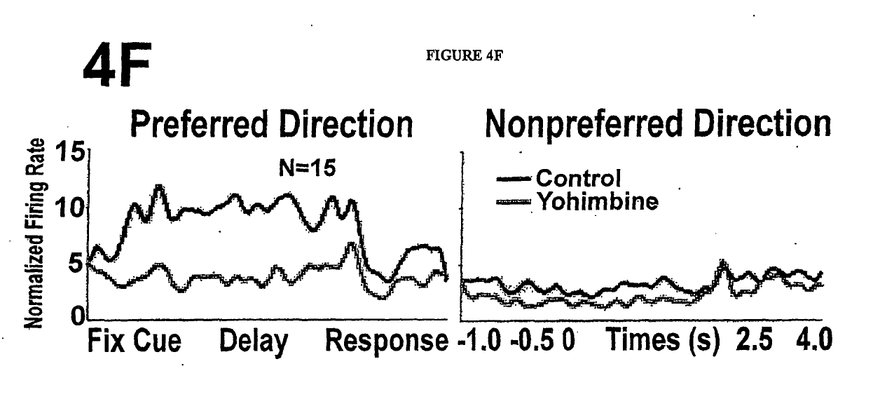

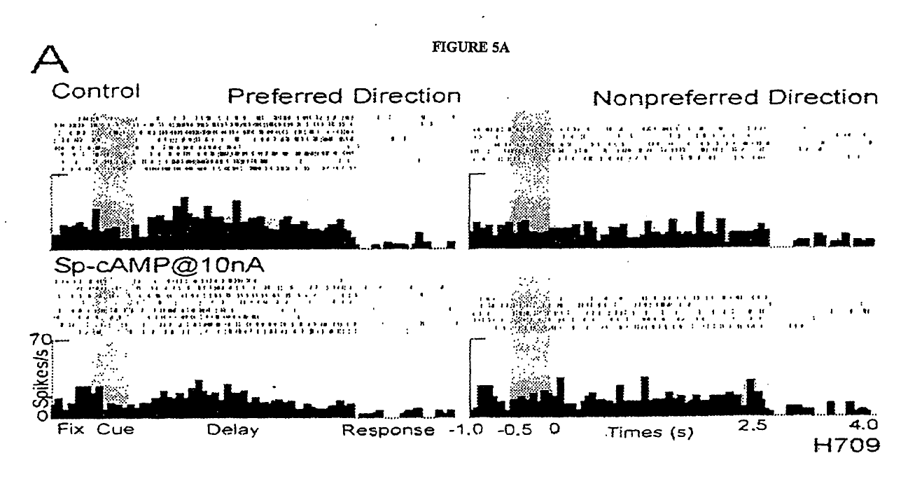

FIG. 4A shows a PFC neuron with relatively weak spatial mnemonic tuning in the control condition. Following guanfacine application (10 nA), delay-related firing was substantially increased for the preferred (one-way ANOVA, P<0.0001) but not for the nonpreferred direction (P>0.05), thus enhancing spatial mnemonic tuning. In neurons with strong spatial mnemonic tuning, the effects of guanfacine (5 nA) were similar but smaller in magnitude, as shown in FIG. 4B (P<0.001). At higher doses, guanfacine (50 nA) did not have enhancing effects (FIG. 4B).

Previous studies demonstrated that iontophoretic application of yohimbine, an α2-AR antagonist, suppressed delay-related firing in PFC neurons (Li et al., 1999). Replicating these previous reports, iontophoresis of yohimbine (15 nA) suppressed delay-related activity for the preferred direction, thus eroding spatial mnemonic tuning (P<0.0001, FIG. 4C). The suppressive effects of yohimbine on delay activity occurred in 15 out of 15 cases (P<0.01 for each cell). Furthermore, co-iontophoresis of yohimbine (15 nA) reversed the enhancing effects of guanfacine on delay-related activity (P<0.01, FIG. 4D), consistent with actions at α2-ARs (population responses for guanfacine and yohimbine alone can be seen in FIGS. 4E and 4F, respectively). These results indicate that endogenous NE stimulation of α2-ARs plays an important role in strengthening delay-related firing.

Influence of cAMP on PFC Neurons Engaged in a WM Task

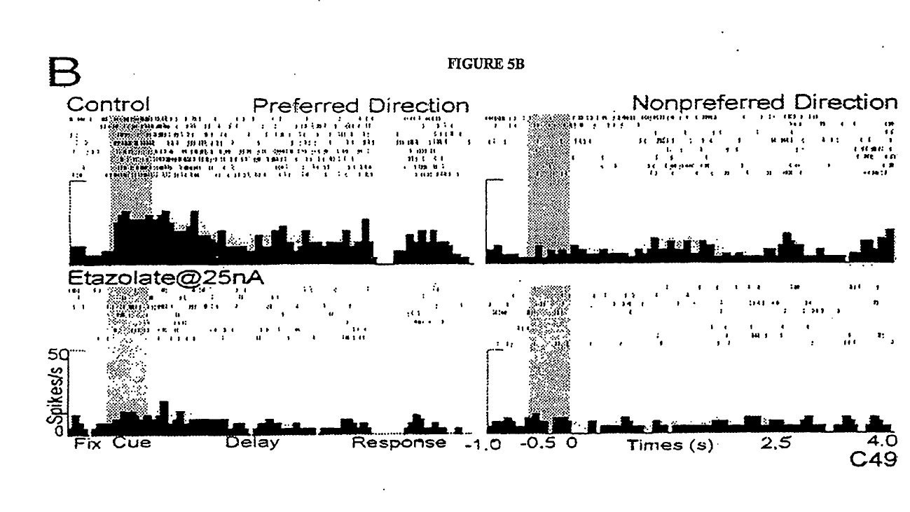

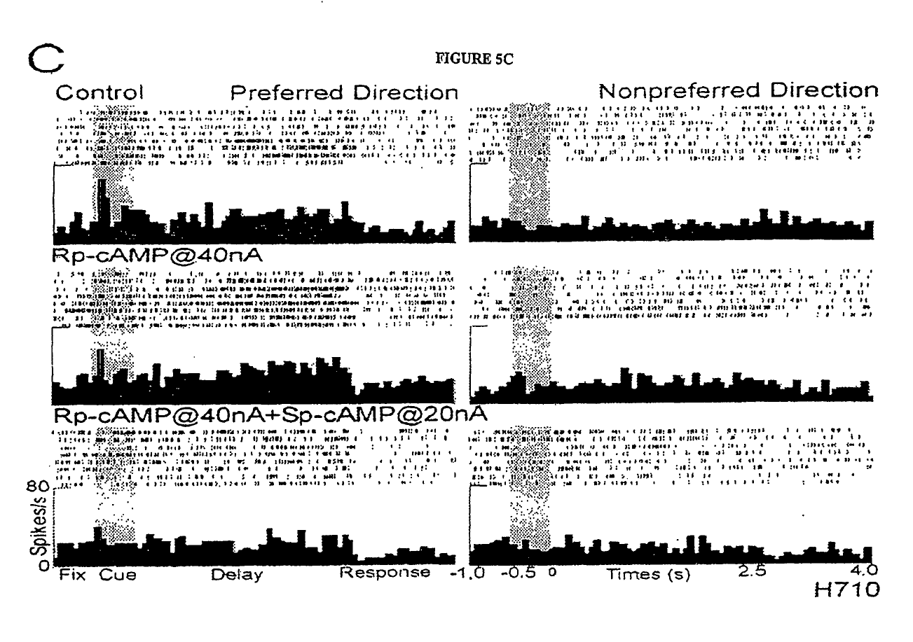

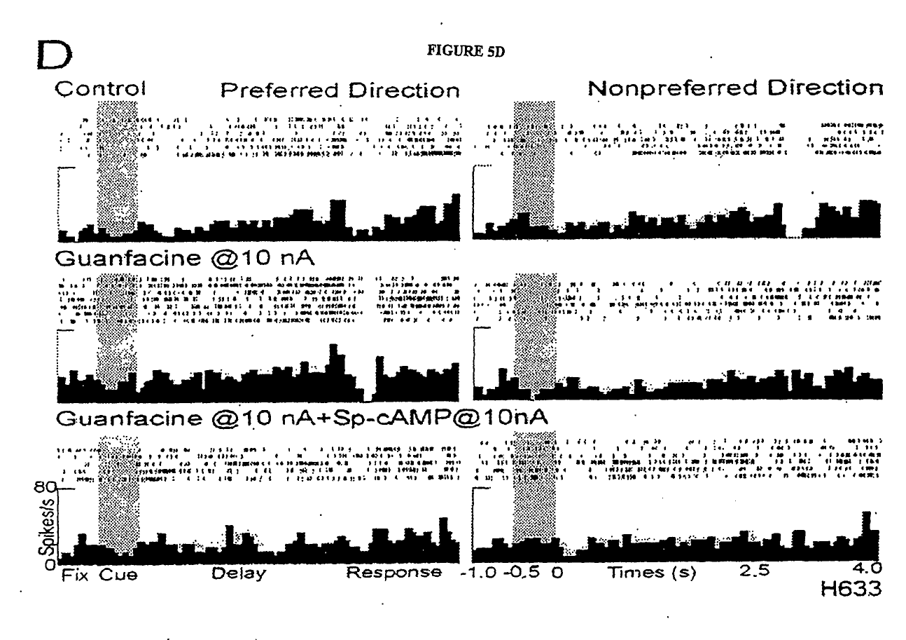

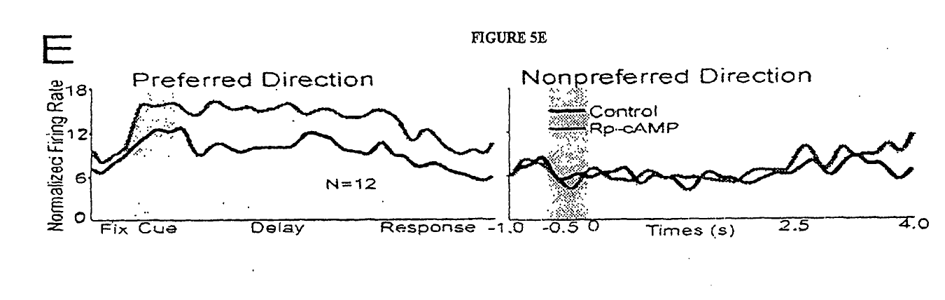

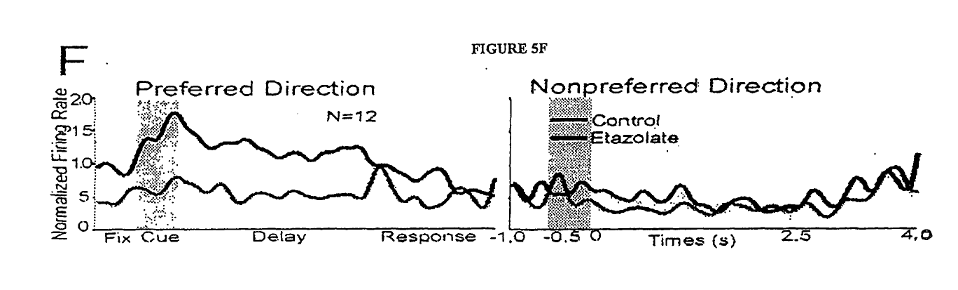

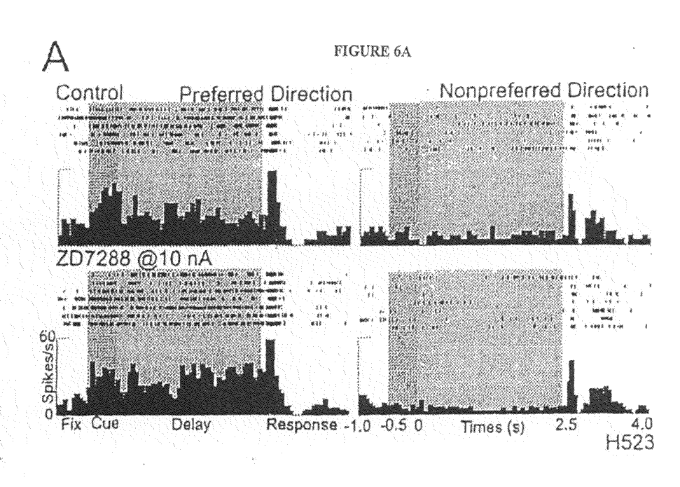

Behavioral studies in rats have shown that amplification of cAMP actions with Sp-cAMPS, a cAMP analog, impaired spatial WM, while inhibition of cAMP actions with Rp-cAMPS ameliorated WM deficits. Consistent with these behavioral results, we found that iontophoresis of Sp-cAMPS (10 nA) significantly decreased delay-related firing for the preferred direction (P<001, FIG. 5A), thus eroding spatial mnemonic tuning. Although the suppressive effects of Sp-cAMPS were replicated in 13 out of 27 neurons, Sp-cAMPS was found to non-specifically increase the neuronal firing in 9 of 27 neurons, and had no effect in 5 neurons. These effects may result from Sp-cAMPS blocking adenosine receptors, inducing a nonspecific increase in firing rate. Thus, we examined an alternative method for increasing cAMP signaling.

Etazolate is a PDE4 inhibitor that increases cAMP levels by inhibiting the breakdown of endogenously produced cAMP. Iontophoretic application of etazolate (25 nA) had highly consistent suppressing effects on spatial mnemonic activity in 10 of 12 PFC neurons. One example is shown in FIG. 5B, in which etazolate dramatically inhibited spatial delay-related firing (P<0.0001). The population response is shown in FIG. 5F. These effects were very rapid, occurring within minutes of application, indicating that endogenous cAMP has powerfuil suppressive effects on PFC neuronal firing.

In contrast to Sp-AMPS and etazolate, iontophoresis of the cAMP inhibitor, Rp-cAMPS (40-50 nA) specifically increased delay-related firing for the preferred direction in 8 of 12 neurons (P<0-001, FIG. 5C, population response in FIG. 5E). This enhancing effect was reversed by subsequent co-iontophoresis of Sp-cAMP (P<0.001), consistent with actions via cAMP signaling. Thus, inhibition of cAMP actions had an effect similar to that of iontophoresis of guanfacine.

Guanfacine Acts Via Inhibition of cAMP

The enhancing effects of guanfacine on WM in rats has been reversed by co-infusion of Sp-cAMPS (Ramos et al., 2006). We examined whether the enhancing effects of guanfacine on PFC neuronal firing are similarly reversed by Sp-cAMPS.

As observed above, iontophoresis of guanfacine (10 nA) significantly increased delay-related firing for the preferred direction (P<0.001, FIG. 5D). Subsequent co-iontophoresis of Sp-cAMPS (10 nA) with guanfacine (10 nA) significantly reversed the enhancing effects of guanfacine in 6 of 9 cases (P<0.001, FIG. 5D). These results are consistent with guanfacine increasing delay-related firing through suppression of cAMP.

### Role of HCN Channels in WM Activity

A putative downstream target of cAMP is the HCN channel. cAMP opens HCN channels, inducing an Ih current which in turn reduces membrane resistance. We examined the role of Ih in WM by iontophoresing the HCN channel blocker, ZD7288.

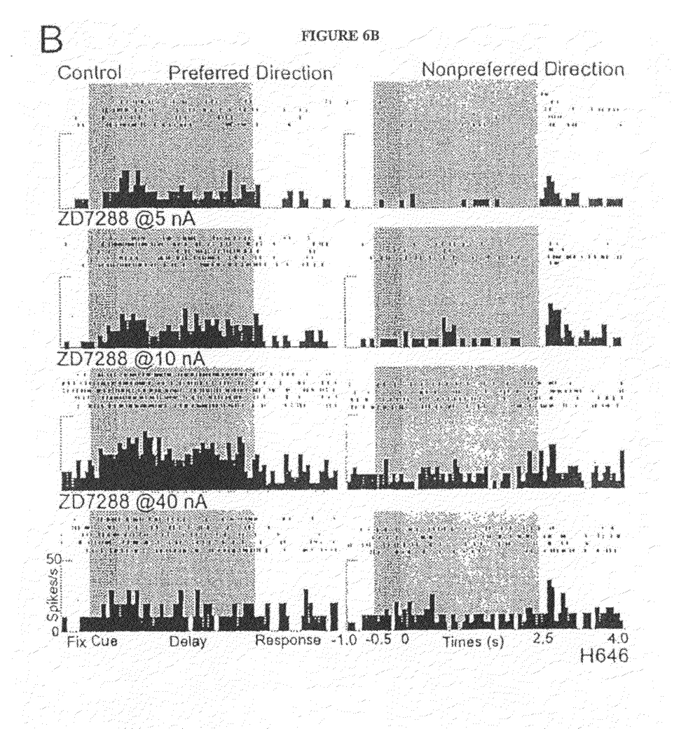

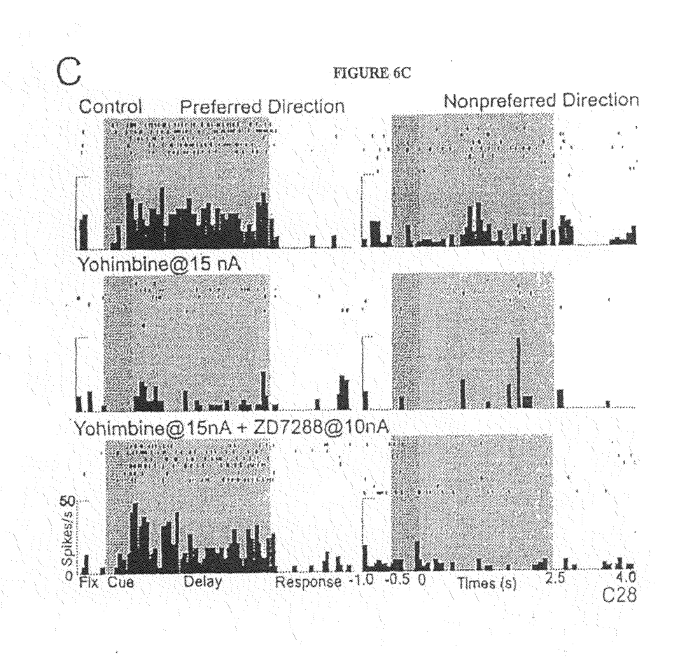

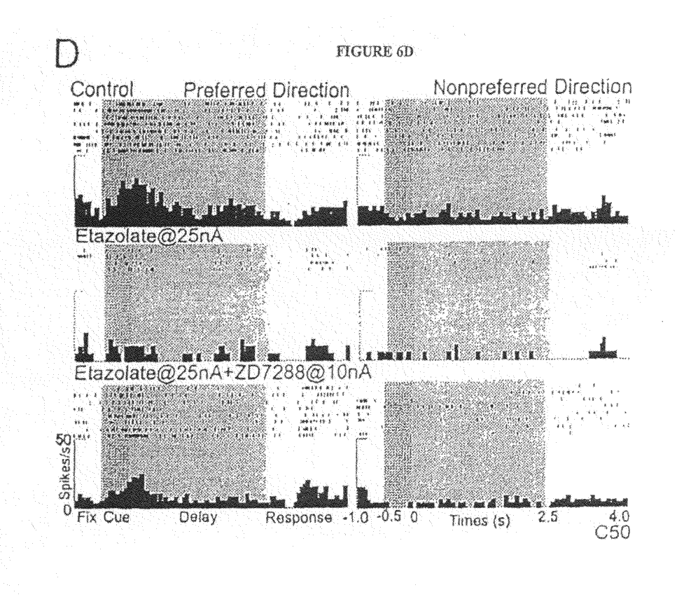

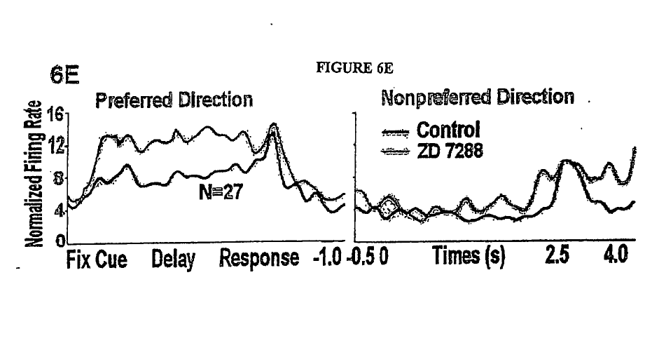

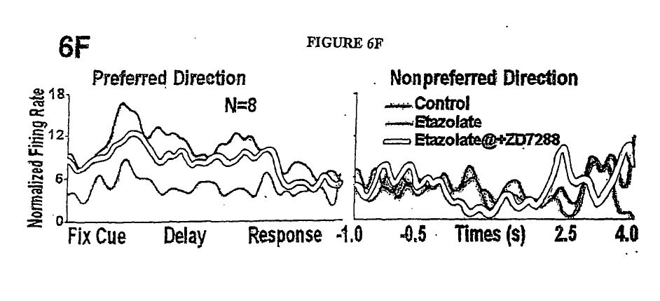

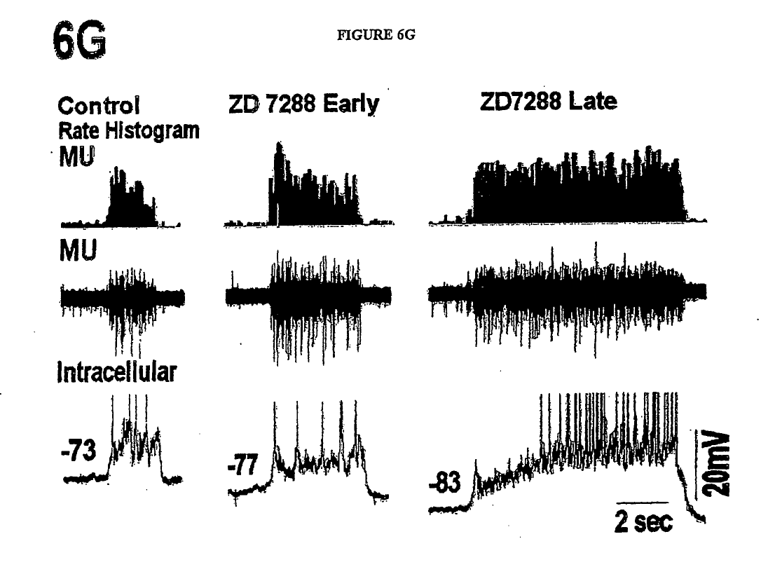

Low doses of ZD7288 (10 nA), like guanfacine or Rp-cAMPS, significantly increased delay-related firing for the preferred direction in 19 of 27 neurons. As with guanfacine, ZD7288 effects were most obvious in neurons with weak spatial tuning, however, small but consistent effects were also observed in well-tuned neurons with robust firing for the preferred direction under control conditions. For example, in FIG. 6A, iontophoretic application of ZD7288 (5 nA) significantly increased delay-related firing for the preferred direction (P<0.001), without altering baseline firing rates (P>0.05), or firing for the nonpreferred direction (P>0.05). ZD7288 had very robust enhancing effects on cells with weak mnemonic firing under control conditions. One example is shown in FIG. 6B, in which application of ZD7288 induced dose-dependent effects, with a low dose (5 nA) significantly increasing delay-related firing for the preferred direction (P<0.001). Subsequent application of ZD7288 at 10 nA further enhanced the delay-related activity for the preferred direction (P<0.0001). This higher dose of ZD 7288 slightly increased the background firing rate, but produced greater delay-related firing for the preferred than the nonpreferred direction (ANOVA on percentage increase, P<0.01). At the highest concentration (40 nA), ZD7288 decreased rather than increased firing (FIG. 6B). These inhibitory effects likely arose from nonspecific blockade of glutamate receptors at high doses (Chen, 2004). The population response for low dose ZD7288 application in 27 neurons is shown in FIG. 6E.

A functional link between HCN channels and α2-ARs was examined by observing whether ZD7288 could reverse the effects of α2-AR blockade (FIG. 6C). As observed above, iontophoretic application of yohimbine (15 nA) dramatically suppressed delay-related activity for the preferred direction (P<0.0001). If this collapse in firing resulted from increased cAMP production and increased numbers of open HCN channels, the yohimbine response should be reversed by co-iontophoresis of ZD7288. Indeed, subsequent co-iontophoresis of ZD7288 significantly reversed the yohimbine response, restoring spatial mnemonic activity (P<0.001). ZD7288 was able to reverse the suppressive effects of yohimbine in 7 of 9 cases (P<0.05 for each case). These data support a functional interaction between α2-ARs and HCN channels at the physiological level.

We also examined the functional interactions between HCN channels and cAMP. As shown in FIG. 6D, the reduction in delay-related firing induced by the PDE4 inhibitor, etazolate (P<0.0001) was fully reversed by co-iontophoresis of ZD7288 (P<0.001). ZD7288 reversed the suppressive effects of etazolate in 6 of 8 cases (P<0.05 for each case; FIG. 4F). Thus, blockade of HCN channels restored delay-related firing under conditions of high endogenous cAMP levels induced by either PDE4 inhibition or α2-AR blockade. These electrophysiological results demonstrate the key role of α2A/cAMP/HCN signaling in permitting spatial mnemonic Sing in PFC networks.

### In Vitro Recordings: Reduction of HCN Activity in PFC Slices Results in Enhanced Recurrent Network Interactions

The cellular and network consequences of modulation of HCN channels was examined with intracellular and whole cell recordings from layer 5 pyramidal cells simultaneously with extracellular multiple unit recordings in the ferret PFC in vitro (n=55, see FIG. 6G). Under normal conditions, these slices generate Up and Down states, consisting of repeating periods of recurrent network activity (Shu et al., 2003). Bath application of ZD7288 (20-50 micromolar; n=10) resulted in a nearly 500% increase in duration of the recurrent network activity of the Up state from an average of 0.84 (±0.25) to 4.1 (±1.3) sec and an increase in Up-state action potential discharge from 1.8±1.7 Hz to 9.2±4.9 Hz (n=9). This ZD7288-induced enhancement of recurrent network activity occurred in conjunction with a progressive hyperpolarization of layer 5 pyramidal neurons (n=12), an increase in apparent input resistance (from 18.6±7.5 to 36±10.8 Mohms, n=10), an increase in the number of action potentials generated by a depolarizing current pulse (not shown), and a marked enhancement of the amplitude of synaptic barrages of the Up state from an average peak amplitude of 7.6±2.7 mV to 20.7±7-2 mV (n=21 FIG. 6G, lowest panel). These results reveal that reduction of the h-current results in significant enhancement of local recurrent network activity in cortical networks, presumably through enhanced effectiveness of dendritic synaptic potentials to initiation action potential activity (Fan et al., 2005; Magee, 1999; Nolan et al., 2004)

### Reduced HCN Channel Activity in Rat PFC Improves Spatial WM Performance

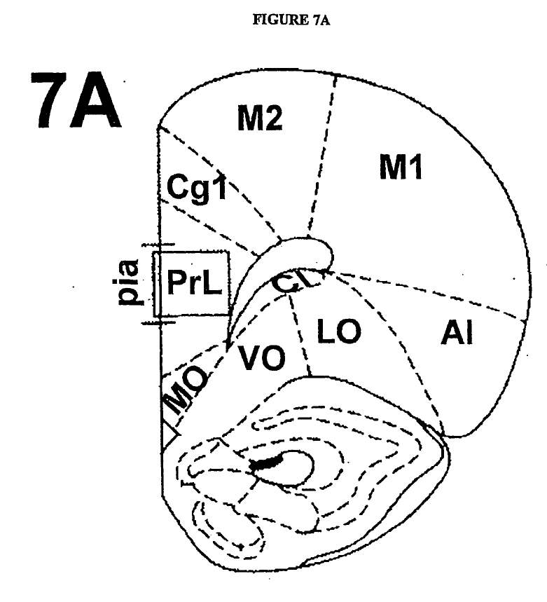

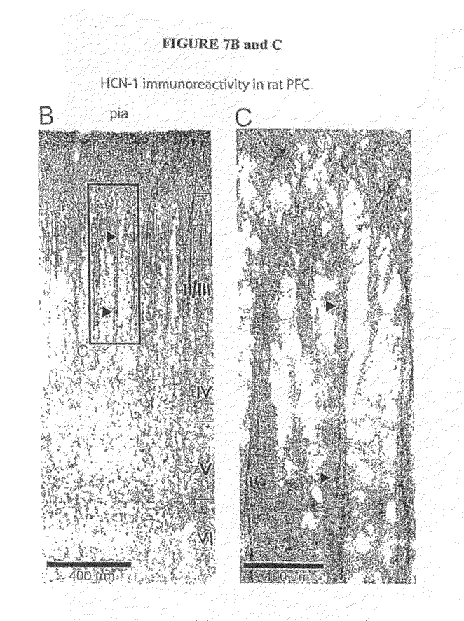

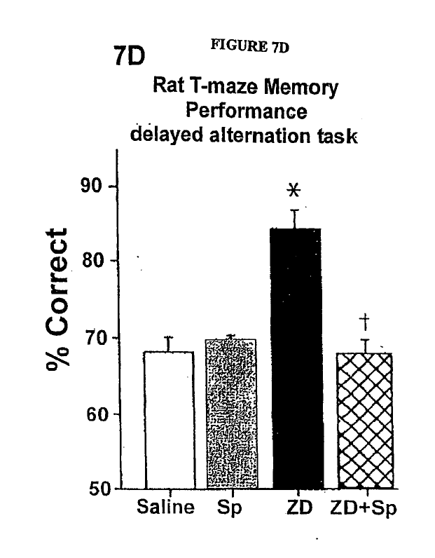

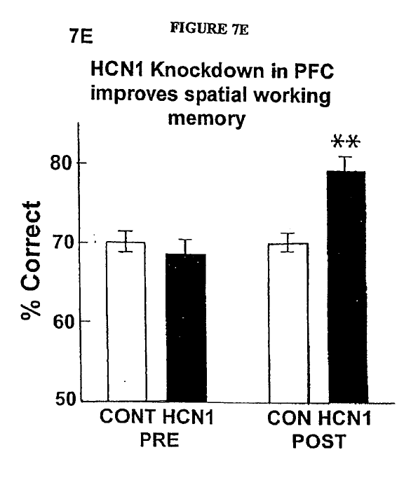

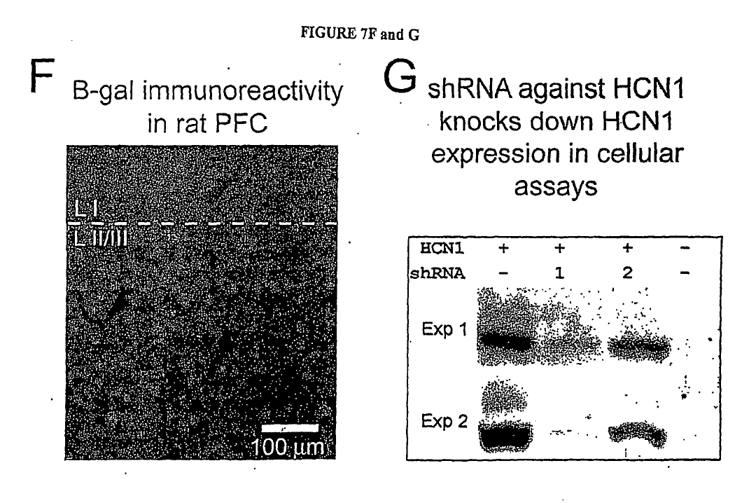

The next study tested the hypothesis that reducing Ih in rat prelimbic PFC (FIG. 7A) would improve performance of a spatial delayed alternation task sensitive to PFC lesions (Larsen and Divac, 1978). The prelimbic PFC displayed strong HCN1 immunoreactivity, with robust labeling of the apical dendrites in the superficial layers (FIGS. 7B, C). Ih was then reduced through either 1) blockade of HCN channels with intra-PFC ZD7288 infusions or 2) knockdown of HCN1 channel expression in PFC.

The first experiment tested whether infusion of ZD7288 into prelimbic PFC would improve performance as previously seen with guanfacine (Ramos et al., 2006) and Rp-cAMPS (Ramos et al., 2003). Pilot experiments explored a wide range of ZD7288 doses (0.00001-0.1 μg/0.5 μl). Improvement was only observed in the low dose range; thus, the 0.00011 g dose became the focus of the present study. ZD7288 was challenged with a low dose of the cAMP analog, Sp-cAMPS (0.21 nmol//0.5 μL) chosen to have no effect on its own. Results are shown in FIG. 7D. Intra-PFC infusion of ZD7288 significantly improved performance, and this effect was blocked by co-infusion of Sp-cAMPS. 2-ANOVA-R analysis showed a significant main effect of ZD7288 infusion, (F(1,4)=23.64, p=0.008); a significant main effect of Sp-cAMPS, (F(1,4)=7.37, p=0.05); and a significant interaction between ZD7288 and Sp-cAMPS (F(1,4)=30.12, p=0.005). Planned comparisons showed that infusion of ZD7288 alone significantly improved accuracy compared to saline (F(1,4)=42.67, p=0.003). Sp-cAMPS infusion had no effect on its own (F(1,4)=1.24, p=0.33), but significantly reversed the enhancing effects of ZD7288 (F(1,4)=18.46, p=0.01; ZD7288+Sp-cAMPS not different than saline: F(1,4)=0.02, p=0.88). Thus, HCN channel blockade enhanced WM at the behavioral and single-cell levels.

In the second experiment, HCN1 expression was knocked down by RNA interference in prelimbic PFC through infusion of one of two short hairpin expressing viral constructs, shRNA-HCN1.1 or shRNA-HCN1 0.2 (FIG. 7G; both were effective and so behavioral results were combined). Performance was compared to rats infused with a scrambled, inactive viral construct (shRNA-scrHCN1). Rats infused with the active constructs showed no change for the first week following infusion (mean % correct±SEM for scrambled: 72.3.4±3.6; for HCN1: 70.2±5.5%; p>0.7), but then showed significant improvements in WM performance 11-19 days after viral infusion. A 2-ANOVA-R analysis showed a significant between subjects effect of viral construct (active vs. scrambled: F(1,6)=9.57, p=0.02), a significant within subjects effect of time (pre- vs. post-infusion performance: F(1,6)=24.61, p=0.003), and a significant interaction between viral construct and time after infusion (F(1,6)=23.5, p=0.003). As shown in FIG. 5E, there was no difference between viral groups at baseline (F(1,6)=1.03, p=0.35), but a significant improvement in the active HCN1 viral group 11-19 days post-infusion (F(1,6)=34.38, p=0.0011). This is consistent with the time course of gene expression from AAV2 that we and others have observed, e.g. (Hommel et al., 2003). Post-mortem analyses confirmed viral transduction of the prelimbic PFC (FIG. 7F) and lack of expression of HCN1 in the rats receiving active construct.

### Electron Microscopy: HCN Channels and α2A-ARs are Co-Localized on Dendritic Spines in Monkey PFC

Ultrastructurally, HCN1 and the pore region of HCN (corresponding to HCN1 and/or HCN2 subunits in the cortex; (Notomi and Shigemoto, 2004) were predominantly detected in dendritic spines and the shafts of pyramidal dendrites (FIGS. 8A, B). Immunoparticles in dendrites were clearly extrasynaptic (i.e. not localized at the active zone or within a 50 nm perisynaptic annulus). In favorable section planes, as in FIG. 8B, HCN channels were observed at the base of emerging spines or, typically, the neck portion. This rather unusual localization could be followed in serial sections and is unlikely to occur as a diffusion artifact, as spine heads in continuity with HCN-immunoreactive necks were often themselves immunonegative. Note that in spine heads, HCN channels appeared both at extrasynaptic membranes and perisynaptically at the edge of asymmetric, presumably excitatory synapses.

α2A-AR-immunoreactive profiles in the neuropil included the spine-laden pyramidal dendrites, besides axonal and glial localization. Distal dendrites showed plasmalemmal but also cytoplasmic labeling, indicative of a rapid turnover from the plasma membrane. Similar to HCN, α2A-AR-immunoparticles in spines marked extrasynaptic and perisynaptic membranes flanking asymmetric synapses. In addition, we observed α2A-AR labeling intracellularly and rarely within the postsynaptic specialization per se. It is worth noting that both the head and the neck portion of spines were immunoreactive for α2A-ARs, as with HCN channels (FIG. 8, compare C to B).

Dual immunolabeling confirmed the co-expression of HCN1 channels and α2A-ARs in spines and dendritic shafts at sites where emerging spines would come into focus. HCN1/α2A-AR labeling involved both the spine head and the neck portion. This pattern was reproduced with reversal of the immunocytochemical sequence, and with both peroxidase/gold (FIGS. 8D, E) and dual gold (FIGS. 8F, G) immunotechniques. Thus, in superficial layers of monkey PFC, HCN1 channels and α2A-ARs are spatially co-expressed on spine membranes.

### Discussion

The current data provide the very first evidence that HCN channels have powerful effects on PFC network firing properties and cognitive performance in animals performing WM tasks. Blockade of HCN channels with ZD7288 promoted persistent network activity and enhanced the spatial mnemonic firing of PFC neurons. Similar enhancing effects were observed with stimulation of α2A-ARs or inhibition of cAMP. Conversely, increasing cAMP signaling—either directly with Sp-cAMPS, or indirectly via blockade of α2-ARs or PDE4 inhibition—dramatically suppressed delay-related firing. Blockade of HCN channels restored mnemonic activity in cells with excessive cAMP signaling induced by either blockade of α2-ARs or PDE4 inhibition, thus demonstrating a functional interaction between α2-ARs, endogenous cAMP, and HCN modulation of PFC neuronal firing. Similar effects were observed at the behavioral level, where HCN channel blockade or HCN1 channel knockdown in PFC improved spatial WM performance. Ultrastructural localization of HCN channels and α2A-ARs indicates that they are ideally situated to modulate synaptic inputs onto PFC pyramidal neurons.

### HCN Channels are Positioned to Gate Cortical-Cortical Inputs in PFC Networks

Imunoelectron microscopy revealed HCN channel expression in spines of pyramidal dendrites in layers I-m of the primate PPC. Gold particles marked both extrasynaptic and perisynaptic membranes flanking asymmetric, presumed excitatory synapses. More interestingly, the spine neck was often HCN-immunoreactive. These data indicate that HCN channels are ideally suited for gating glutamatergic transmission mediated by axospinous synapses, including cortico-cortical inputs of the superficial PFC. When opened in the presence of cAMP, HCN channels would lower membrane resistance and shunt inputs to the spine. Due to the small cytosolic volume, HCN channels in the spine head—and especially those in the spine neck—are capable of “sensing” minute neurochemical changes in the milieu caused by the downstream effects of “co-localized” neurotransmitter receptors. Our ultrastructural data suggest that one such candidate receptor in PFC is the α2A-AR. It is noteworthy that the latter was found not only in the head but also in the neck of spines on both perisynaptic and extrasynaptic membranes, and dual labeling confirmed the spatial co-expression of HCN1 and α2A-ARs. α2A-ARs inhibit cAMP production via Gi signaling and thus could have a powerfuil influence on the open-state of nearby HCN channels. Inadequate stimulation of α2A-ARs would result in elevated cytosolic cAMP levels, opening Ih channels, and selectively disconnecting axospinous inputs to cortical pyramids. Therefore, cytosol compartmentalization in the spine and the co-expression of HCN channels with α2A-ARs on spine membranes may provide a cellular basis for altering the strength of excitatory transmission at individual axospinous synapses, thus modulating circuit connectivity in PFC.

α2A-AR Inhibition of cAMP-HCN Signaling Enhances the Spatially Tuned, Delay-Related Firing of PFC Pyramidal Cells

The functional implications suggested by immunoelectron microscopy were supported by our physiological data. Delay-related firing arises from reverberating, excitatory circuits in PFC, and depends on the functional connectivity of neurons with shared spatial tuning. The current study found that agents, which 1) stimulate α2A-ARs, 2) inhibit cAMP signaling or 3) block HCN channels, all increase delay-related firing for the preferred direction, consistent with increased functional connectivity under conditions where HCN channels are closed. Conversely, delay-related firing collapsed in the presence of agents that blocked α2A-ARs or increased cAMP signaling, consistent with reduced functional connectivity when cAMP opens HCN channels. These modulatory effects were very powerful: extremely low doses selectively altered delay-related firing for the preferred direction, while slightly higher doses had more generalized effects on firing, consistent with perturbations in network firing. Parallel results were observed at the behavioral level, where PFC infusions of guanfacine (Ramos et al., 2006) or ZD7288, or knockdown of HCN1 expression in PFC, all improved WM performance, while agents that blocked α2A-ARs (Li and Mei, 1994) or accentuated cAMP signaling (Taylor et al., 1999) impaired WM. It is remarkable to have such confluence between behavioral and electrophysiological findings.

ZD7288 is currently the most selective HCN antagonist available, and as such, has become the standard pharmacological method for assessing Ih mechanisms, e.g. (Fan et al., 2005). However, it has recently been noted that at higher doses ZD7288 becomes nonselective, producing ALA and NMDA glutamate receptor blockade (Chen, 2004). The inhibition of cell activity at higher ZD7288 concentrations (25-40 nA) in the current study could be consistent with reduced glutamate receptor excitation. A similar profile was observed in our behavioral data, where pilot studies showed that higher doses of ZD7288 were ineffective, whereas a very low dose consistently improved WM performance.

Physiological interactions between α2A-ARs and HCN channels were observed, consistent with their co-expression in spines: The collapse in memory fields induced by the α2A-AR antagonist, yohimbine, was reversed by the HCN channel blocker, ZD7288. HCN channel blockade similarly reversed the suppressive effects of the PDE4 inhibitor, etazolate. Thus, iontophoresis of yohimbine, Sp-cAMPS or etazolate, all induced an immediate collapse in memory fields, likely due to a reduction in recurrent excitatory drive in the PFC network. The blockade of HCN channels with ZD7288 restored a normal firing pattern. It should be noted that these agents are not likely competing at the same individual channel; rather, it is likely that ZD7288 and Sp-cAMPS (or cAMP per se) each alter the open state of a subset of HCN channels, and we record the integration of this population response. It is striking that very low doses can have such rapid and robust effects on cell firing, revealing a powerful mechanism for the dynamic regulation of PFC microcircuits.

### Comparison to HCN Actions on Long-Term Memory Functions of the Hippocampus and Cerebellum

Recent studies of HCN1 knockout mice have revealed distinct roles of Ih in cerebellum and hippocampus, and it is instructive to compare the current results with these important findings. In cerebellar Purkinje cells, HCN1 channels mediate an inward current that stabilizes the integrative properties of the cell and ensures that their input-output function is independent of previous activity (Nolan et al., 2003). HCN1 currents allow Purkinje cells to integrate information very quickly, consistent with the rapid time scale of cerebellar mechanisms (ibid). However, the influence of HCN channels in cerebellum occurs only when Purkinje cells are hyperpolarized. In contrast, pyramidal cells in hippocampus and PFC have a lower resting membrane potential, and thus HCN channels may play a role under resting conditions in these cells (Nolan et al., 2004). Pyramidal cells in PFC and hippocampus share other properties: HCN1 channels are localized on their distal dendrites, and, when opened, appear to shunt synaptic inputs onto those distal locations (Magee, 1999; Nolan et al., 2004). However, there are important differences between CA1 and PFC neurons as well. In hippocampus, high levels of cAMP are essential for long-term potentiation and memory consolidation, leading to long-lasting changes in synaptic architecture (cAMP also plays a beneficial role when hippocampus interacts with PFC under conditions when delay lengths are very long, see (Runyan and Dash, 2005). In contrast, the WM operations of the PFC depend on the transient activation of microcircuits that are disrupted by high levels of cAMP. Thus, cAMP/HCN signaling may play an especially important role in PFC.

### Model of α2A-cAMP HCN Modulation of PFC Networks

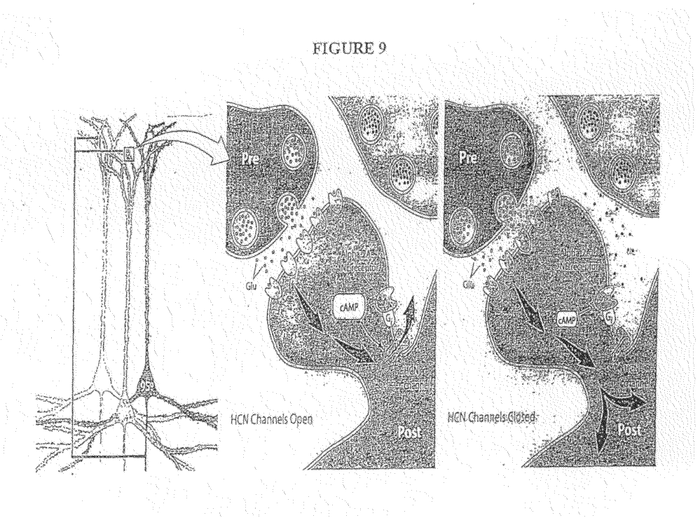

FIG. 9 illustrates a model whereby HCN channels on spines of PFC distal pyramidal dendrites modulate the efficacy of excitatory inputs. Pyramidal cells form reverberating circuits through mutual, axospinous excitatory connections. HCN channels are localized on the spine neck or near excitatory synapses on spine head membranes. When HCN channels are open in the presence of cAMP, they pass Ih which lowers membrane resistance and effectively shunts synaptic input, reducing the functional connectivity of the network (FIG. 9A). NE regulates this process. We have shown α2A-ARs and HCN1 channels on the same spine membranes. Stimulation of α2A-ARs, e.g. with guanfacine, inhibits the production of cAMP, closing HCN channels and increasing the efficacy of synaptic inputs, thus strengthening the functional connectivity of PFC microcircuits (FIG. 9B). Thus, α2A/cAMP/HCN signaling provides a mechanism for dynamically regulating the strength of PFC networks.

The examples further indicate that Gs-coupled receptors may temporarily suppress neurotransmission by activating cAMP production, opening HCN channels and shunting synaptic inputs. For example, dopamine D1 receptors (D1Rs) are coupled to Gs and are also concentrated on spines in superficial PFC (Smiley et al., 1994). We have recently observed that moderate levels of D1R stimulation suppress firing for nonpreferred spatial directions via a cAMP-mediated mechanism (Vijayraghavan et al., 2007). It is not known if this suppression involves opening of HCN channels. If so, it is possible that α2A-ARs amplify inputs for preferred directions by selectively closing HCN channels on spines receiving inputs from neurons with shared spatial preferences, while D1Rs may shunt inputs from neurons tuned to nonpreferred directions. Thus, the open state of HCN channels may determine which pattern of microcircuits are functionally connected at any one time to appropriately regulate behavior and thought based on immediate cognitive demands.

### Relevance of α2A-AR-cAMP-HCN Signaling to Arousal State and Stress

αa2A/cAMP/HCN signaling in superficial cortical layers likely regulates the strength of PFC function based on the animal's state of arousal. Low levels of NE cell firing during drowsy conditions (Aston-Jones et al., 1999) may lead to insufficient NE stimulation of α2A-ARs, inadequate inhibition of cAMP, and impaired WM. Conversely, exposure to uncontrollable stress impairs WM via excessive catecholamine release (Arnsten, 2000). Similar effects are observed at the cellular level, where memory fields collapse under neurochemical conditions induced by stress: e.g. etazolate, Sp-cAMPS, (FIG. 3) or high levels of D1R stimulation activating cAMP (Vijayraghavan et al., 2007). Thus, under conditions of uncontrollable stress, cortico-cortical connections of superficial PFC would be functionally disconnected, rendering the PFC “de-corticate”. This process may be exacerbated in patients with alterations in genes that regulate cAMP signaling, e.g. COMT (Egan et al., 2001) or DISC1 (Millar et al., 2005), increasing vulnerability to PFC dysfunction in illnesses such as schizophrenia that are worsened by stress exposure. Intriguingly, DISC1 protein has been localized to dendritic spines in human PFC, (Kirkpatrick et al., 2006), suggesting that it may normally regulate Ih, but may inadequately suppress cAMP levels in spines of patients with schizophrenia Guanfacine has recently been shown to strengthen PFC cognitive function in patients with schizotypal disorder (McClure et al., 2006), as well as those with ADHD (Scahil et al., 2001; Taylor and Russo, 2001). The present data suggest that some of these enhancing effects may result from reduced cAMP production and the closure of HCN channels. These results represent the first time that we have understood the mechanism of action of a psychotropic medication at the level of an ion channel, and illustrate the powerful influence of arousal pathways on cortical microcircuitry.

The previously described examples demonstrate that HCN1 or HCN1/HCN2 heteromers in spines of pyramidal dendrites in the superficial layers of primate PFC are spatially co-expressed with the α2A-AR, thus providing a potent substratum for functional interaction. Electrophysiological and cognitive experiments support a model where α2A-AR agonists such as guanfacine improve PFC cognitive function by inhibiting the production of cAMP, closing HCN channels, and strengthening the PFC networks that underlie delay-related cell firing in monkeys performing a spatial WM task.

These data indicate that the enhancing effects of guanfacine result from closure of Ih channels via inhibition of cAMP production. Given that guanfacine is currently in use for the treatment of neuropsychiatric and neurologic disorders involving weakened prefrontal cortical function—Attention Deficit Hyperactivity Disorder and Tourettes Syndrome (Scahill et al, Amer J Psychiatry, 158:1067-1074, 2001), mild traumatic brain injury (McAllister et al, Brain Inj, 18:331-350 2004), schizophrenia-related illness (Friedman et al, Guanfacine treatment of cognitive impairment in schizophrenia A pilot study. Neuropsychopharmacology, 2001), post-traumatic stress disorder (Horrigan, J Amer Acad Child Adol Psychiatry, 35:975-976, 1996)—these data indicate that agents according to the present invention that block Ih channels in the prefrontal cortex would have therapeutic effects.

It is to be understood by those skilled in the art that the foregoing description and examples are illustrative of practicing the present invention, but are in no way limiting. Variations of the detail presented herein may be made without departing from the spirit and scope of the present invention as defined by the following claims.

### REFERENCES

Aoki, C., Venkatesan, C., Go, C.-G., Forman, R., and Kurose, H. (1998). Cellular and subcellulal sites for noradrenergic action in the monkey dorsolateral prefrontal cortex as revealed by the immunocytochemical localization of noradrenergic receptors and axons. Cerebral Cortex 8, 269-277.
Arnsten, A. F. T. (2000). Stress impairs PFC function in rats and monkeys: Role of dopamine D1 and norepinephrine alpha-1 receptor mechanisms. Prog Brain Res 126, 183-192.
Arnsten, A. F. T., Cai, J. X., and Goldman-Rakic, P. S. (1988). The alpha-2 adrenergic agonist guanfacine improves memory in aged monkeys without sedative or hypotensive side effects. J Neurosci 8, 4287-4298.
Arnsten, A. F. T., and Goldman-Rakic, P. S. (1985). Alpha-2 adrenergic mechanisms in prefrontal cortex associated with cognitive decline in aged nonhuman primates. Science 230, 1273-1276.
Aston-Jones, G., Rajkowski, J., and Cohen, J. (1999). Role of locus coeruleus in attention and behavioral flexibility. Biological Psychiatry 46, 1309-1320.
Auricchio, A., Hildinger, M., O'Connor, E., Gao, G. P., and Wilson, J. M. (2001). Isolation of highly infectious and pure adeno-associated virus type 2 vectors with a single-step gravity-flow column. Hum Gene Ther 12, 71-76.
Chen, C. (2004). ZD7288 inhibits postsynaptic glutamate receptor-mediated responses at hippocampal perforant path-granule cell synapses. Eur J Neurosci 19:643-649.
Chen, S., Wang, J., and Siegelbaum, S. A. (2001). Properties of hyperpolarization-activated pacemaker current defined by coassembly of HCN1 and HCN2 subunits and basal modulation by cyclic nucleotide. J Gen Physiol 117, 491-504.
Egan, M. F., Goldberg, T. E., Kolachana, B. S., Callicott, J. H., Mazzanti, C. M., Straub, R. E., Goldman, D., and Weinberger, D). R. (2001). Effect of COMT Val108/158 Met genotype on frontal lobe function and risk for schizophrenia. Proc Natl Acad Sci USA 98, 6917-6922.
Fan, Y., Fricker, D., Brager, D., Chen, X, Lu, H. C., Chitwood, R. A., and Johnston, D. (2005 Activity-dependent decrease of excitability in rat hippocampal neurons through increases in I(h). Nat Neurosci 8, 1542-1551.
Franowicz, J. S., Kessler, L., Dailey-Boja, C. M., Kobilka, B. K., Limbird, L. E., and Arnsten, A. F. T. (2002). Mutation of the alpha2A-adrenoceptor impairs working memory performance and annuls cognitive enhancement by guanfacine. J Neurosci 22, 8771-8777.
Fuster, J. M. (1985). The prefrontal cortex, mediator of cross-temporal contingencies. Human Neurobiol 4, 169-179.
Goldman-Rakic, P. S. (1995). Cellular basis of working memory. Neuron 14, 477-485.
Goldman-Rakic, P. S., Lidow, M. S., and Gallager, D. W. (1990). Overlap of dopaminergic, adrenergic, and serotonergic receptors and complementarity of their subtypes in primate prefrontal cortex. J Neurosci 10, 2125-2138.
Hommel, J. D., Sears, R. M., Georgescu, D., Simmons, D. L., and DiLeone, R. J. (2003). Local gene knockdown in the brain using viral-mediated RNA interference. Nat Med 9, 1539-1544.
Jakala, P., Riekkinen, M., Sirvio, J., Koivisto, E., Kejonen, K, Vanhanen, M., and Riekkinen, P. J. (1999). Guanfacine, but not clonidine, improves planning and working memory performance in humans. Neuropsychopharmacology 20, 460-470.
Kirkpatrck, B., Xu, L., Cascella, N., Ozeki, Y., Sawa, A., and Roberts, R. C. (2006). DISC1 immunoreactivity at the light and ultrastructural level in the human neocortex. J Comp Neurol 497, 436-450.
Kritzer, M. F., and Goldman-Rakic, P. S. (1995). Intrinsic circuit organization of the major layers and sublayers of the dorsolateral prefrontal cortex in the rhesus monkey. J Comp Neurol 359, 131-143.
Larsen, J. K, and Divac, 1. (1978). Selective ablations within the prefrontal cortex of the rat and performance of delayed alternation. Physiolog Psychol 6, 15-17.
Li, B.-M., Mao, Z.-M., Wang, M., and Mei Z.-T. (1999). Alpha-2 adrenergic modulation of prefrontal cortical neuronal activity related to spatial working memory in monkeys. Neuropsychopharmacol 21, 601-610.
Li, B.-M., and Mei, Z.-T. (1994). Delayed response deficit induced by local injection of the alpha-2 adrenergic antagonist yohimbine into the dorsolateral prefrontal cortex in young adult monkeys. Behav Neural Biol 62, 134-139.
Lörincz, A., Notomi, T., Tamas, G., Shigemoto, R., and Nusser, Z. (2002). Polarized and compartment-dependent distribution of HCN1 in pyramidal cell dendrites. Nat Neurosci 5, 1185-1193.
Magee, J. C. (1999). Dendritic Ih normalizes temporal summation in hippocampal CA1 neurons. Nat Neurosci 2, 508-514.
Mao, Z.-M., Arnsten, A. F. T., and Li, B.-M. (1999). Local infusion of alpha-i adrenergic agonist into the prefrontal cortex impairs spatial working memory performance in monkeys. Biol Psychiatry 46, 1259-1265.
McAllister, T. W., Flashman, L. A., Sparling, M. B., and Saykin, A. J. (2004). Working memory deficits after traumatic brain injury: catecholaminergic mechanisms and prospects for treatment—a review. Brain Inj 18, 331-350.
McClure, M: M., Barch, D. M., Romero, M. 3., Harvey, P. D., and Siever, L. J. (2006). The effects of guanfacine on context processing abnormalities in schizotypal personality disorder. Biological Psychiatry in press.
Millar, J. K, Pickard, B. S., Mackie, S., James, R. S., Christie, S., Buchanan, S. R., Malloy, M. P., Chubb, J. E., Huston, E., Baillie, G. S, et al. (2005). DISC1 and PDE4B are interacting genetic factors in schizophrenia that regulate cAMP signaling. Science 310, 1187-1191.
Miller, E. K., Erickson, C. A, Desimone, R., and (1996). Neural mechanisms of visual working memory in prefrontal cortex of the macaque. J Neurosci 16, 5154-5167.
Nolan, M. F., Malleret, G., Dudman, J. T., Buhl, D. L., Santoro, B., Gibbs, E., Vronskaya, S., Buzsaki, G., Siegelbaum, S. A., Kandel, E. R., and Morozov, A. (2004). A behavioral role for dendritic integration: HCN1 channels constrain spatial memory and plasticity at inputs to distal dendrites of CA1 pyramidal neurons. Cell 119, 719-732.
Nolan, M. F., Malleret, G., Lee, K. H., Gibbs, E., Dudman, J. T., Santoro, B., Yin, D., Thompson R. F., Siegelbaum, S. A., Kandel, E. R., and Morozov, A. (2003). The hyperpolarization-activated HCN1 channel is important for motor learning and neuronal integration by cerebellar Purkinje cells Cell 115, 551-564.
Notomi, T., and Shigemoto, R. (2004). Immunohistochemical localization of Ih channel subunits, HCN1-4, in the rat brain. J Comp Neurol 471, 241-276.
Paxinos, G., and Watson, C. (1997). The rat brain in stereotaxic coordinates, 3rd edn (New York: Academic Press).
Ramos, B., Birnbaum, S. B., Lindenmayer, I., Newton, S. S., Duman, R., and Arnsten, A. F. T. (2003). Dysregulation of protein kinase A signaling in the aged prefrontal cortex: New strategy for treating age-related cognitive decline. Neuron 40, 835-845.
Ramos, B., Stark, D., Verduzco, L., van Dyck, C. H., and Arnsten, A. F. T. (2006). Alpha-2A-Adrenoceptor stimulation improves prefrontal cortical regulation of behavior through inhibition of cAMP signaling in aging animals. Learning and Memory 13, 770-776.
Runyan, J. D., and Dash, P. K. (2005). Distinct prefrontal molecular mechanisms for information storage lasting seconds versus minutes. Learn Mem 12, 232-238.
Scahill, L., Chappell, P. B., Kim, Y. S., Schultz, R. T., Katsovich, L., Shepherd, E., Arnsten, A. F. T., Cohen, D. J., and Leckman, J. F. (2001). Guanfacine in the treatment of children with tic disorders and ADHD: A placebo-controlled study. Amer J Psychiatry 158, 1067-1074.
Shu, Y.-S., Hasenstaub, A., Badoual, M., Bal, T., and McCormick, D. A. (2003). Barrages of synaptic activity control the gain and sensitivity of cortical neurons. J Neurosci 23, 10388-10401.
Smiley, J. F., Levey, A. I., Ciliax, B. J., and Goldman-Rakic, P. S. (1994). D1 dopamine receptor immunoreactivity in human and monkey cerebral cortex: predominant and extrasynaptic localization in dendritic spines. Proc Natl Acad Sci USA 91, 5720-5724.
Taylor, F. B., and Russo, J. (2001). Comparing guanfacine and dextroamphetamine for the treatment of adult Attention Deficit-Hyperactivity Disorder. J Clin Psychopharm 21, 223-228.
Taylor, J. R., Birnbaum, S. G., Ubriani, R., and Arnsten, A. F. T. (1999). Activation of protein kinase A in prefrontal cortex impairs working memory performance. I Neuroscience (Online) 19RC23.
Ulens, C., and Tytgat, J. (2001). Functional heteromerization of HCN1 and HCN2 pacemaker channels. J Biol Chem 276, 6069-6072.
Vijayraghavan, S., Wang, M., Birnbaum, S. G., Bruce, C. J., Williams, G. V., and Arnsten, A. F. T. (2007). Inverted-U dopamine D1 receptor actions on prefrontal neurons engaged in working memory. Nature Neuroscience In press.
Wang, M., Vijayraghavan, S., and Goldman-Rakic, P. S. (2004). Selective D2 receptor actions on the functional circuitry of working memory. Science 303, 853-856.

1. A method of treating a cognitive or mood disorder in a patient comprising administering to said patient an effective amount of a compound which is a blocker or inhibitor of Iₕ channels in said patient and optionally, an effective amount of guanfacine or its pharmaceutically acceptable salt form and/or chelerythrine in its neutral or salt form.

2. The method according to claim 1 wherein said Iₕ channels are found in the prefrontal cortex of said patient and comprise a HCN1/HCN2 heteromer.

3. The method according to claim 1 wherein said cognitive disorder is a memory or learning disorder.

4. The method according to claim 1 wherein said cognitive disorder or mood disorder is attention deficit disorder (ADD), attention deficit disorder with hyperactivity (ADD-HD), autism, pervasive development disorder, learning disability, major depressive disorder, seasonal affective disorder, bipolar disorder, unipolar disorder, post-partum depression or schizophrenia.

5. The method according to claim 1 wherein said cognitive disorder results from aging, trauma, stroke, neurodegenerative disorders, drug-induced states, neurotoxic agents, aging or anxiety disorders.

6. The method according to claim 1 wherein said blocking or inhibitor compound is a compound according to the structure: 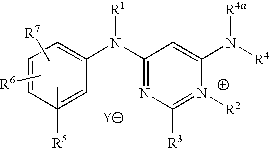
Where R¹ is H, or an optionally substituted C₁-C₃ alkyl, preferably a C₂ alkyl (ethyl) group;
R² is an optionally substituted C₁-C₃ alkyl group, preferably a methyl group;
R³ is H, an optionally substituted C₁-C₃ alkyl (preferably methyl), a halogen or O(C₁-C₃) alkyl;
R⁴ is an optionally substituted C₁-C₆ alkyl, C(O)—(C₁-C₅)alkyl, C(O)-aryl, C(O)O—(C₁-C₄)alkyl, C(O)O-aryl, or an optionally substituted heterocyclic, aryl or heteroaryl group;
R^{4a} is H or an optionally substituted C₁-C₆ (preferably a C₁-C₃) alkyl;
R⁵, R⁶ and R⁷ are each independently H, halogen, an optionally substituted C₁-C₆ alkyl (preferably, an optionally substituted C₁-C₃ alkyl), O—(C₁-C₃) alkyl, optionally substituted heterocyclic, aryl or heteroaryl group;
Y⁻ is an anion of a pharmaceutically acceptable salt (a physiologically acceptable anion, preferably a Cl⁻, Br⁻, I⁻, OAc⁻);
or a solvate or polymorph thereof, optionally, in combination with a pharmaceutically acceptable carrier, additive or excipient.

7. The method according to claim 1 wherein said inhibitor or blocker compound is

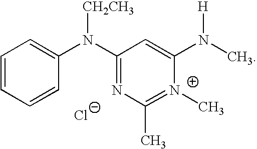

8. A method of enhancing cognitive function in a patient comprising administering to said patient an effective amount of a blocker or inhibitor of Iₕ channels in said patient.

9. The method according to claim 1 wherein said Iₕ channels are found in the prefrontal cortex of said patient and comprise a HCN1/HCN2 heteromer.

10. The method according to claim 8 wherein said cognitive function is memory or learning.

11. The method according to claim 8 wherein said blocking or inhibitor compound is a compound according to the structure: 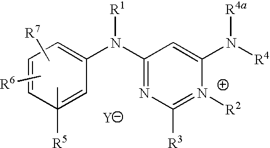
Where R¹ is H, or an optionally substituted C₁-C₃ alkyl, preferably a C₂ alkyl (ethyl) group;
R² is an optionally substituted C₁-C₃ alkyl group, preferably a methyl group;
R³ is H, an optionally substituted C₁-C₃ alkyl (preferably methyl), a halogen or O(C₁-C₃) alkyl;
R⁴ is an optionally substituted C₁-C₆ alkyl, C(O)—(C₁-C₅)alkyl, C(O)-aryl, C(O)O—(C₁-C₄)alkyl, C(O)O-aryl, or an optionally substituted heterocyclic, aryl or heteroaryl group;
R^{4a} is H or an optionally substituted C₁-C₆ (preferably a C₁-C₃) alkyl;
R⁵, R⁶ and R⁷ are each independently H, halogen, an optionally substituted C₁-C₆ alkyl (preferably, an optionally substituted C₁-C₃ alkyl), O—(C₁-C₃) alkyl, or an optionally substituted heterocyclic, aryl or heteroaryl group;
Y⁻ is an anion of a pharmaceutically acceptable salt (a physiologically acceptable anion, preferably a Cl⁻, Br⁻, I⁻, OAc⁻);
or a solvate or polymorph thereof, optionally, in combination with a pharmaceutically acceptable carrier, additive or excipient.

12. The method according to claim 1 wherein said inhibitor or blocker compound is

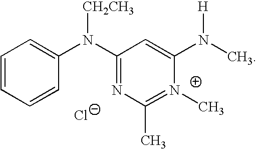

13. The method according to claim 1 wherein said inhibitor or blocker is coadministered with guanfacine or its pharmaceutically acceptable salt.

14. The method according to claim 1 wherein said inhibitor or blocker is coadministered with chelerythrine in its neutral or salt form.

15. The method according to claim 1 wherein said inhibitor or blocker is coadministered with both guanfacine or its pharmaceutically acceptable salt and chelerythrine or its salt form.

16.-32. (canceled)

33. A compound according to the structure: 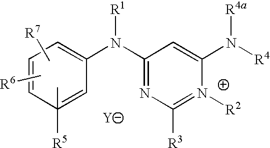
Where R¹ is H, or an optionally substituted C₁-C₃ alkyl, preferably a C₂ alkyl (ethyl) group;
R² is an optionally substituted C₁-C₃ alkyl group, preferably a methyl group;
R³ is H, an optionally substituted C₁-C₃ alkyl (preferably methyl), a halogen or O(C₁-C₃) alkyl;
R⁴ is an optionally substituted C₁-C₆ alkyl, C(O)—(C₁-C₅)alkyl, C(O)-aryl, C(O)O—(C₁-C₄)alkyl, C(O)O-aryl, or an optionally substituted heterocyclic, aryl or heteroaryl group;
R^{4a} is H or an optionally substituted C₁-C₆ (preferably a C₁-C₃) alkyl;
R⁵, R⁶ and R⁷ are each independently H, halogen, an optionally substituted C₁-C₆ alkyl (preferably, an optionally substituted C₁-C₃ alkyl), O—(C₁-C₃) alkyl, optionally substituted heterocyclic, aryl or heteroaryl group;
Y⁻ is an anion of a pharmaceutically acceptable salt (a physiologically acceptable anion, preferably a Cl⁻, Br⁻, I⁻, OAc⁻);
or a solvate or polymorph thereof, optionally, in combination with a pharmaceutically acceptable carrier, additive or excipient.

34. A compound according to claim 33 which is

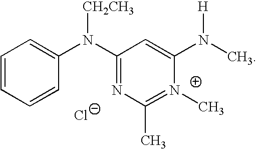

35. A pharmaceutical composition comprising an effective amount of a compound according to claim 33 in combination with a pharmaceutically acceptable carrier, additive or excipient.

36. A pharmaceutical composition comprising an effective amount of a compound according to claim 34 in combination with a pharmaceutically acceptable carrier, additive or excipient.

37. A pharmaceutical composition comprising an effective amount of a compound according to claim 33 in combination with an effective amount of guanfacine or its pharmaceutically acceptable salt and a pharmaceutically acceptable carrier, additive or excipient.

38. A pharmaceutical composition comprising an effective amount of a compound according to claim 33 in combination with an effective amount of chelerythrine in its neutral or salt form and a pharmaceutically acceptable carrier, additive or excipient.

39. A pharmaceutical composition comprising an effective amount of a compound according to claim 33 in combination with an effective amount of guanfacine or its pharmaceutically acceptable salt form, chelerythrine in its neutral or salt form and a pharmaceutically acceptable carrier, additive or excipient.

40. A method of treating the loss of function in patients from cAMP opening of Iₕ (HCN) channels and prefrontal cortical impairment (PFC) comprising administering to said patient an effective amount of an Iₕ channel blocker or inhibitor.

41. The method according to claim 40 wherein said loss results in a mood disorder or schizophrenia.

42. The method according to claim 41 wherein said mood disorder is major depressive disorder, seasonal affective disorder, bipolar disorder, unipolar disorder or post-partum depression.

43. The method according to claim 40 wherein said blocker or inhibitor is a compound according to the structure: 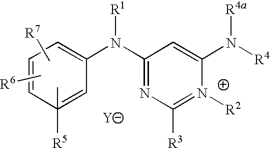
Where R¹ is H, or an optionally substituted C₁-C₃ alkyl, preferably a C₂ alkyl (ethyl) group;
R² is an optionally substituted C₁-C₃ alkyl group, preferably a methyl group;
R³ is H, an optionally substituted C₁-C₃ alkyl (preferably methyl), a halogen or O(C₁-C₃) alkyl;
R⁴ is an optionally substituted C₁-C₆ alkyl, C(O)—(C₁-C₅)alkyl, C(O)-aryl, C(O)O—(C₁-C₄)alkyl, C(O)O-aryl, or an optionally substituted heterocyclic, aryl or heteroaryl group;
R^{4a} is H or an optionally substituted C₁-C₆ (preferably a C₁-C₃) alkyl;
R⁵, R¹ and R⁷ are each independently H, halogen, an optionally substituted C₁-C₆ alkyl (preferably, an optionally substituted C₁-C₃ alkyl), O—(C₁-C₃) alkyl, optionally substituted heterocyclic, aryl or heteroaryl group;
Y⁻ is an anion of a pharmaceutically acceptable salt (a physiologically acceptable anion, preferably a Cl⁻, Br⁻, I⁻, OAc⁻), or a solvate or polymorph thereof, optionally, in combination with a pharmaceutically acceptable carrier, additive or excipient.

44.-46. (canceled)
